(19) United States
(12) Patent Application Publication
(10) Pub. No.: US 2025/0011369 A1
(43) Pub. Date: Jan. 9, 2025

(54) Cyclopeptide Glass and Pharmaceutical Composition Glass Containing Cyclopeptide

(71) Applicant: Institute of Process Engineering, Chinese Academy of Sciences, Beijing (CN)

(72) Inventors: Xuehai Yan, Beijing (CN); Ruirui Xing, Beijing (CN); Chengqian Yuan, Beijing (CN)

(73) Assignee: Institute of Process Engineering, Chinese Academy of Sciences, Beijing (CN)

(21) Appl. No.: 18/702,025

(22) PCT Filed: Nov. 5, 2021
(86) PCT No.: PCT/CN2021/129065

§ 371 Date: Apr. 17, 2024

(51) Int. Cl. C07K 7/66; A61K 38/00; A61P 31/04; C07K 5/12; C07K 7/58; C07K 9/00

(52) U.S. Cl. CPC C07K 7/66; A61P 31/04; C07K 5/12; C07K 7/58; C07K 9/008; A61K 38/00

(57) ABSTRACT

The present invention discloses a cyclopeptide glass and a pharmaceutical composition glass containing a cyclopeptide. The cyclopeptide glass of the present invention can simultaneously exert efficacy and function as a drug adjuvant. Compared with crystals and traditional drug dosage forms or adjuvants, the cyclopeptide glass effectively increases a drug dissolution rate, improves drug bioavailability, and may be widely used in the fields of drug delivery and sustained release for resistance to tumors, resistance to viruses/bacteria, blood sugar control, immune regulation, neuromodulation, etc.

### TECHNICAL FIELD

The present invention belongs to the technical field of medicine and relates to new dosage forms and new adjuvants of cyclopeptide drugs, and in particular to a cyclopeptide glass and a cyclopeptide pharmaceutical composition glass. The present invention further involves related technologies for effectively utilizing poorly soluble drugs, and particularly relates to a method for preparing a glassy drug by converting a poorly soluble cyclopeptide drug into a glassy state. This cyclopeptide glass may be used in the fields of drug delivery for resistance to tumors, resistance to viruses/bacteria, blood sugar control, immune regulation, neuromodulation, etc., especially to achieve sustained release of drugs.

### BACKGROUND

A cyclopeptide is formed by condensation and cyclization of a plurality of amino acids through peptide bonds, and has a structure with certain conformational constraints. Unique topological structures of cyclopeptides make them exceptionally stable to chemical, thermal, and biological degradation. There are a large number of hydrogen bond acceptors and donors in the cyclopeptides, and hydrogen bonding is one of the main ways for drugs to interact with receptors. Therefore, the cyclopeptide often has certain biological and pharmacological activities and has become an important pharmacophore in medicinal chemistry. In addition, restricted conformations of the cyclopeptides enable them to exhibit better specificity and targeting affinity than linear peptides. Therefore, the cyclopeptides exhibit a wide range of biological and pharmacological activities, such as resistance to tumors, viruses, bacteria and aging, immunomodulation, memory enhancement, blood sugar regulation, etc.

However, the significant rigid structure and strong hydrogen bond-forming ability of cyclopeptide molecules lead to their poor water solubility, easy crystallization, and low bioavailability, which seriously affect their medicinal activities. At present, domestic and foreign researchers have adopted a variety of pharmaceutics methods to improve the above shortcomings, including several strategies including direct chemical modification of cyclopeptides (such as N-methylation modification) or nanocarrier encapsulation (such as lipids or micelles). However, problems such as complex synthesis processes, potential toxicity, and low loading of cyclopeptides are still inevitable.

Compared with crystals, which are more thermodynamically stable, a glassy state is a metastable amorphous structure that can retain the biological and pharmacological activities of cyclopeptide molecules. Compared with crystalline cyclopeptide drugs, glassy cyclopeptide drugs exhibit high surface free energy and high dispersion, which can effectively increase a dissolution rate and bioavailability of the cyclopeptides.

It should be noted in particular that a cyclopeptide glass of the present invention can completely replace traditional pharmaceutical adjuvants and achieve functions such as dispersion, solubilization, adhesion, and controlled release. In particular, it can be used as a substitute for pharmaceutical adjuvants such as castor oil to effectively avoid severe allergic reactions caused by histamine.

There have been no public reports on a cyclopeptide glass, especially on pharmaceutical composition glass with biological/pharmacological activities and sustained-release functions, and on a method for converting poorly soluble cyclopeptides into a glassy state.

Surprisingly, the present invention has found that cyclopeptides and their derivatives can be processed into a cyclopeptide glass and a cyclopeptide pharmaceutical composition glass through specific preparation processes. The present invention also discovers a method for converting a poorly soluble cyclopeptide drug into a glassy state, which effectively increases a dissolution rate and bioavailability of cyclopeptide drugs.

The present invention is completed based on this discovery. The cyclopeptide glass discovered based on the present invention is expected to be used as an active drug or pharmaceutical adjuvant, and may be widely used in the fields of drug delivery and sustained release for resistance to tumors, resistance to viruses/bacteria, blood sugar control, immunomodulation, neuromodulation, etc.

### SUMMARY

A primary objective of the present invention is to provide a cyclopeptide glass and a cyclopeptide pharmaceutical composition glass, and a method for converting a poorly soluble cyclopeptide into a glassy state. In the present invention, the glass refers to an amorphous solid showing a glass transition phenomenon; the glassy state refers to a disordered organizational structure that maintains glass-like characteristics, has no crystalline structure in X-ray diffraction detection, and has a definite glass transition temperature. That is, the present invention provides a method for converting a poorly soluble cyclopeptide from a crystalline state to a glassy state, thus effectively improving an in vitro dissolution rate and solubility of the poorly soluble cyclopeptide, and improving the bioavailability of a poorly soluble cyclopeptide drug.

In a first aspect: a cyclopeptide-based glass, wherein the cyclopeptide is a cyclopeptide having a structural formula 1 or a salt thereof, and the cyclopeptide is one or a combination of two or more of cyclopeptides,

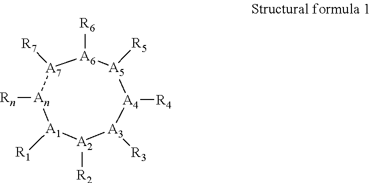

A₁-Aₙ are independently selected from one or a combination of more of:
glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, selenocysteine and pyrrolysine;
R₁-Rₙ are independently selected from the group consisting of H and other modifiable groups, preferably, the modifiable groups are independently selected from the group consisting of methyl, alkyl, phosphoric acid, acetyl, formyl, fatty acid, benzoyl, amide, ester, 9-fluorenylmethyloxycarbonyl, and tert-butoxycarbonyl;
n≥2, preferably, 2≤n≤15, A₁-Aₙ are linked by amino acid condensation.

Preferably, the cyclopeptide has a biological and/or pharmacological activity; and further preferably, the cyclopeptide has antibacterial/antiviral, anti-tumor, blood sugar regulating, and immunomodulatory activities, wherein
the cyclopeptide having an antibacterial/antiviral activity includes, but is not limited to the following structures and similar structures thereof:

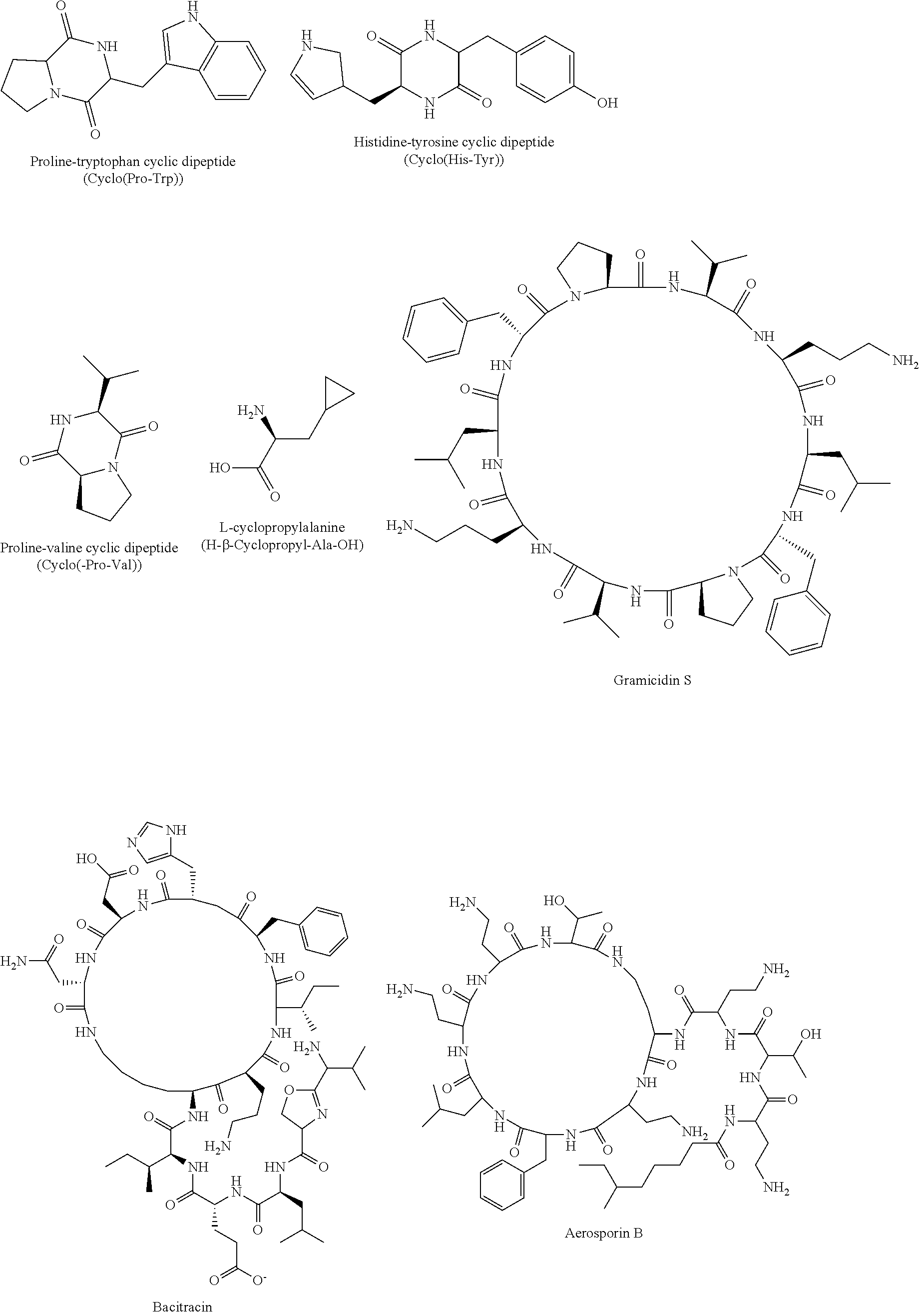

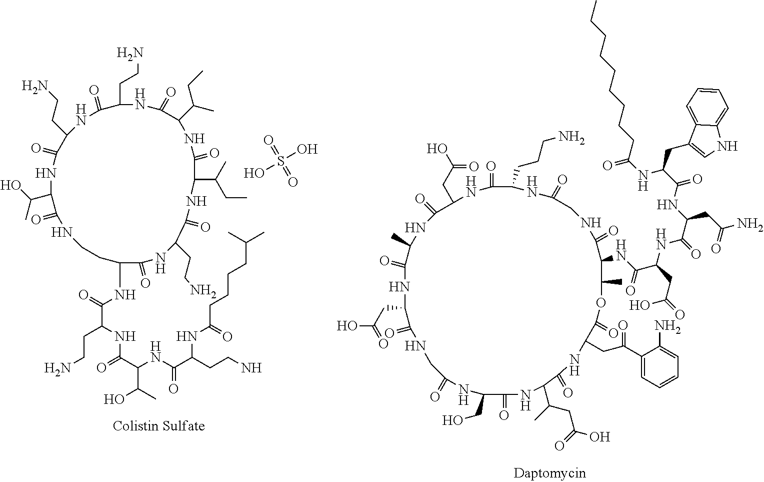

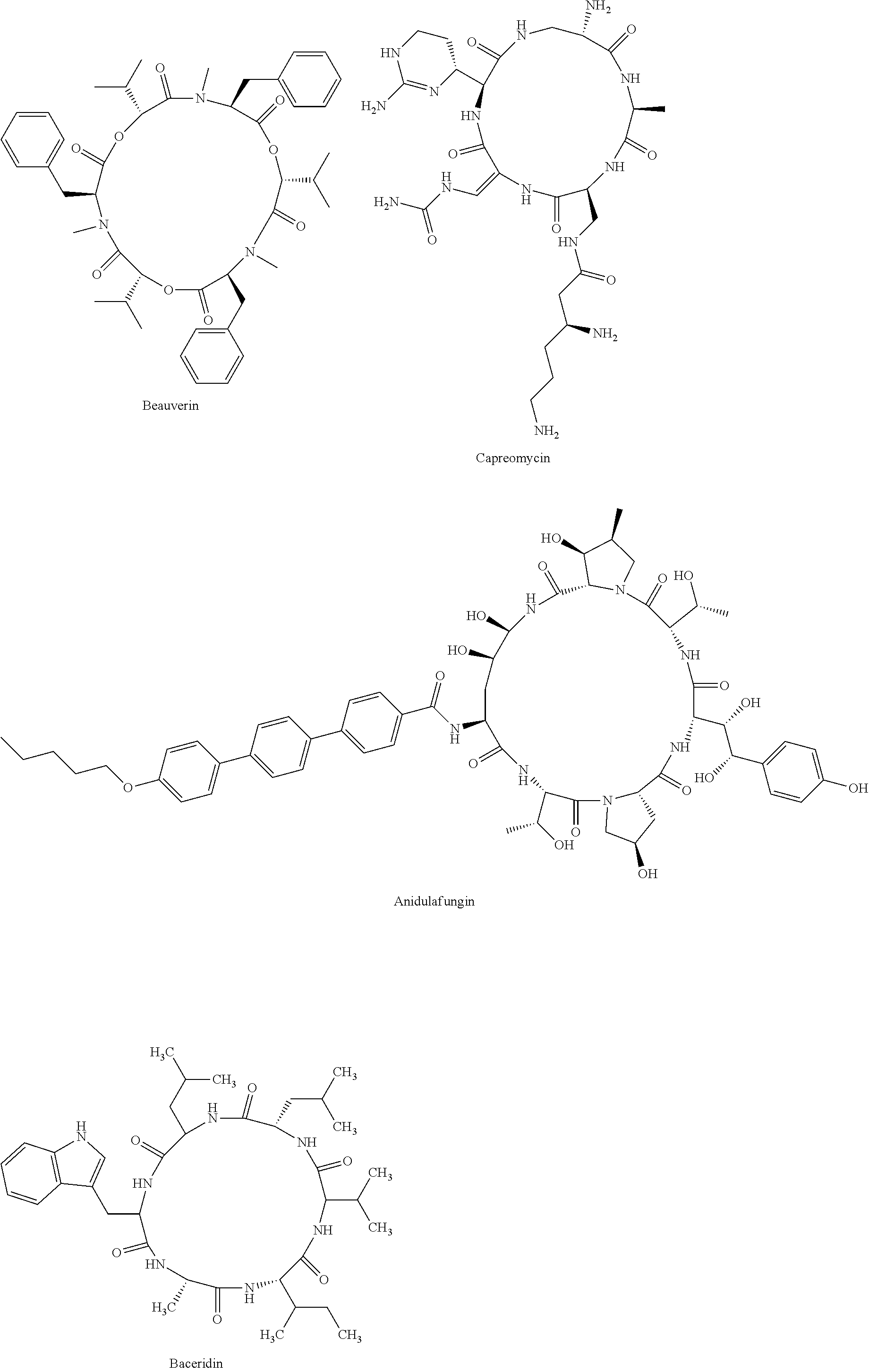

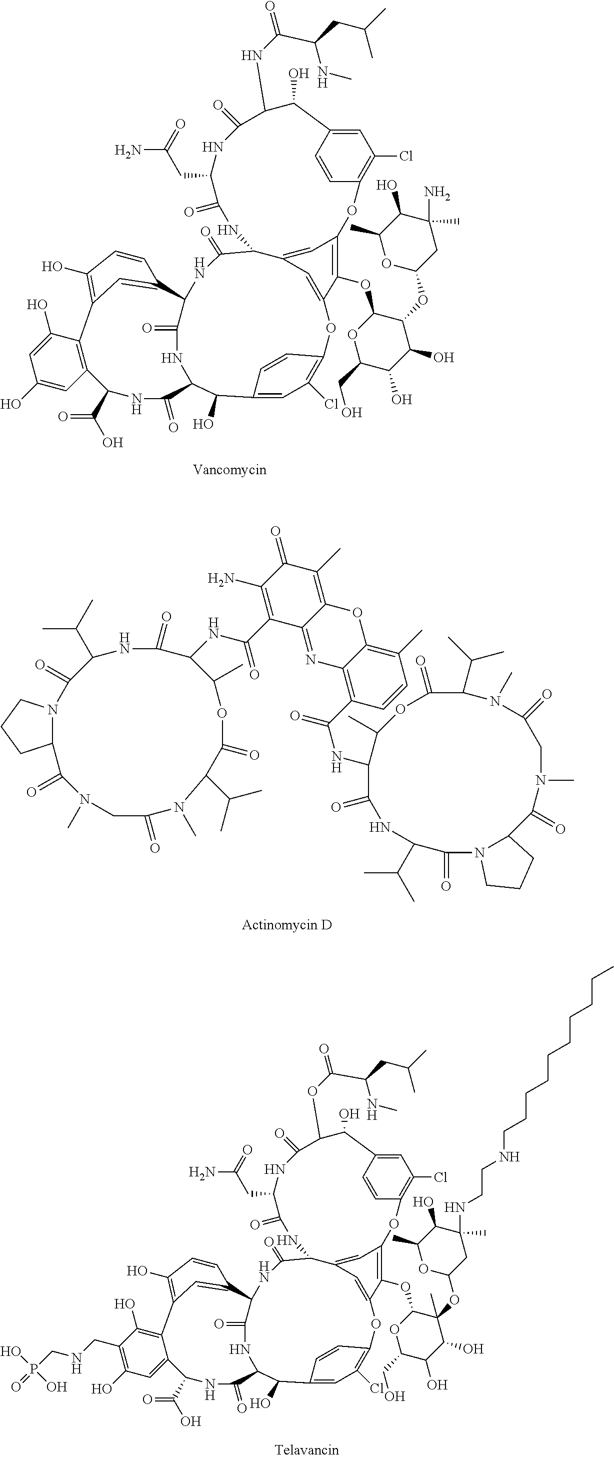

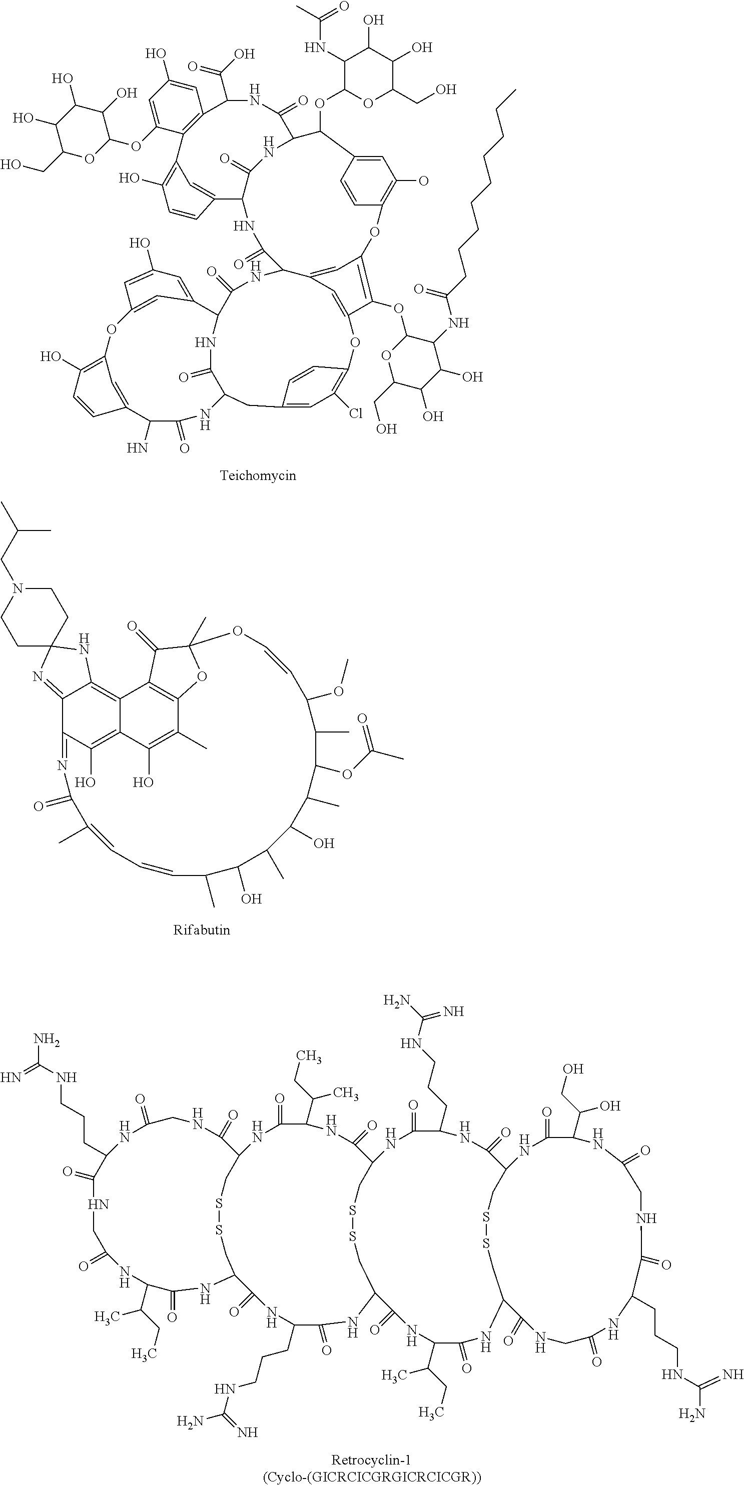

the cyclopeptide having an anti-tumor activity includes, but is not limited to, the following structures and similar structures thereof:

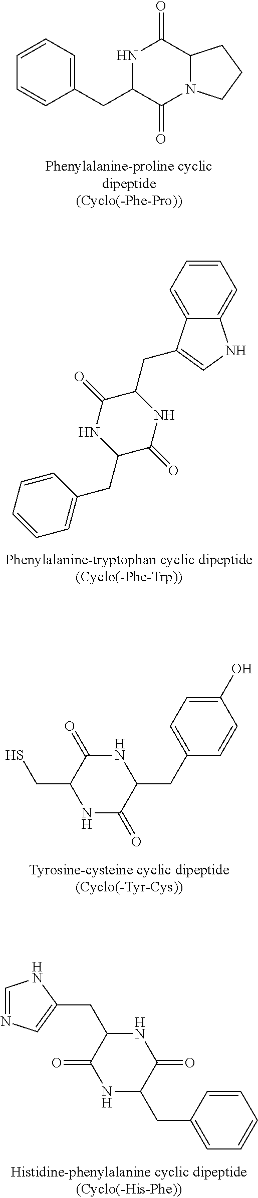 [SMILES: O=C1NC(Cc2ccccc2)C(=O)N2CCCC12 ; O=C1NC(Cc2c[nH]c3ccccc23)C(=O)NC1Cc1ccccc1 ; O=C1NC(Cc2ccc(O)cc2)C(=O)NC1CS ; O=C1NC(Cc2cnc[nH]2)C(=O)NC1Cc1ccccc1]

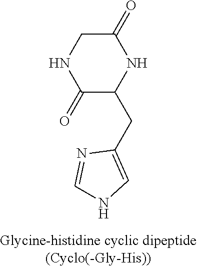 [SMILES: O=C1CNC(=O)C(Cc2c[nH]cn2)N1]

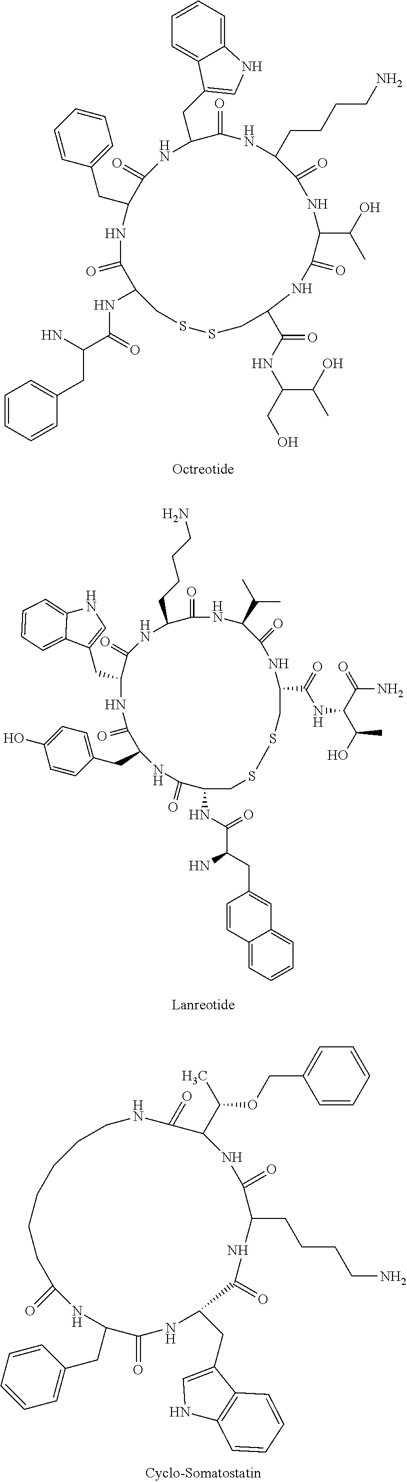 [SMILES: CC(O)C(CO)NC(=O)C1CSSCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1 ; CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccc3ccccc3c2)CSSC[C@@H](C(=O)N[C@H](C(N)=O)[C@@H](C)O)NC1=O ; C[C@H](OCc1ccccc1)C1NC(=O)C(CCCCN)NC(=O)[C@H](Cc2c[nH]c3ccccc23)NC(=O)C(Cc2ccccc2)NC(=O)CCCCCCNC1=O]

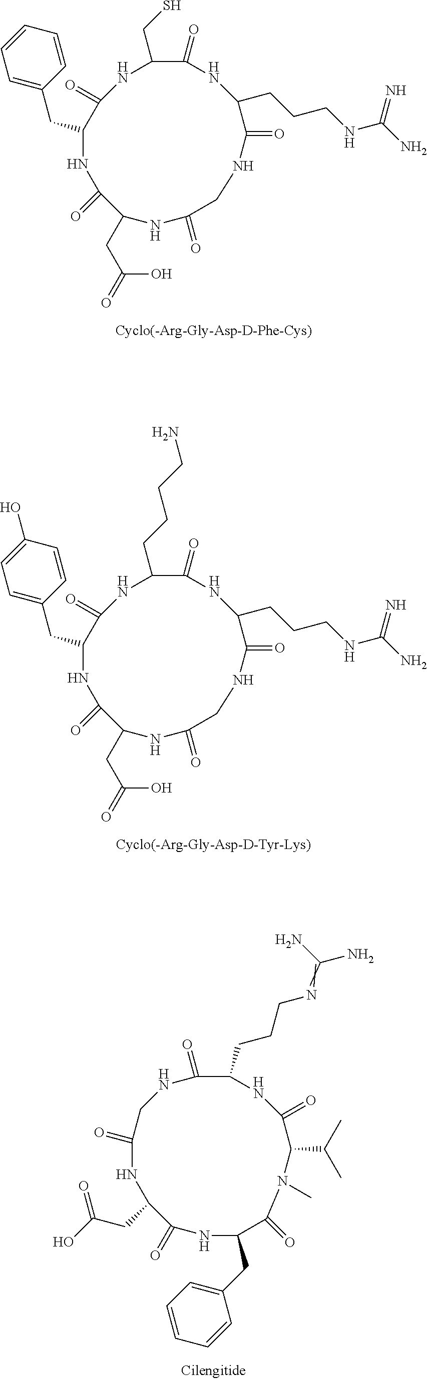

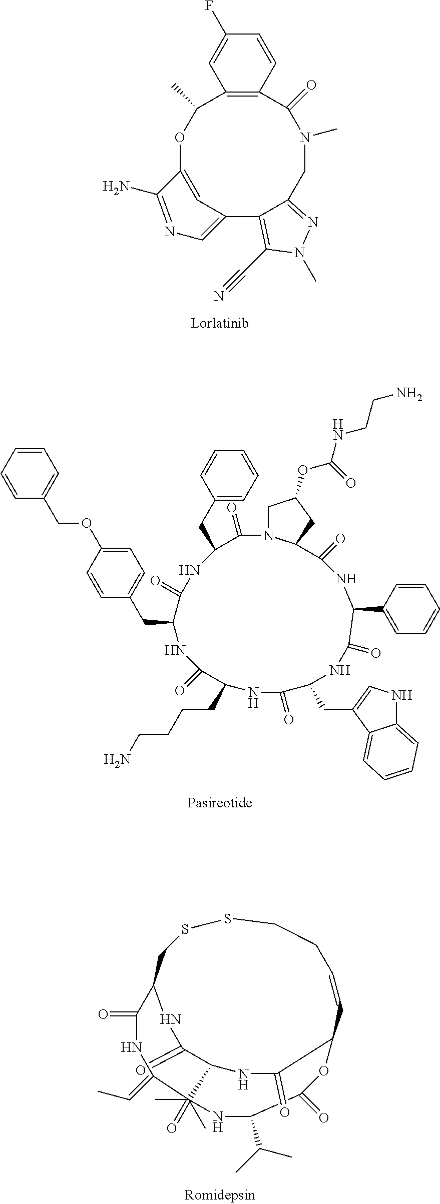

the cyclopeptide having an immunomodulatory activity includes, but is not limited to, the following structures and similar structures thereof:

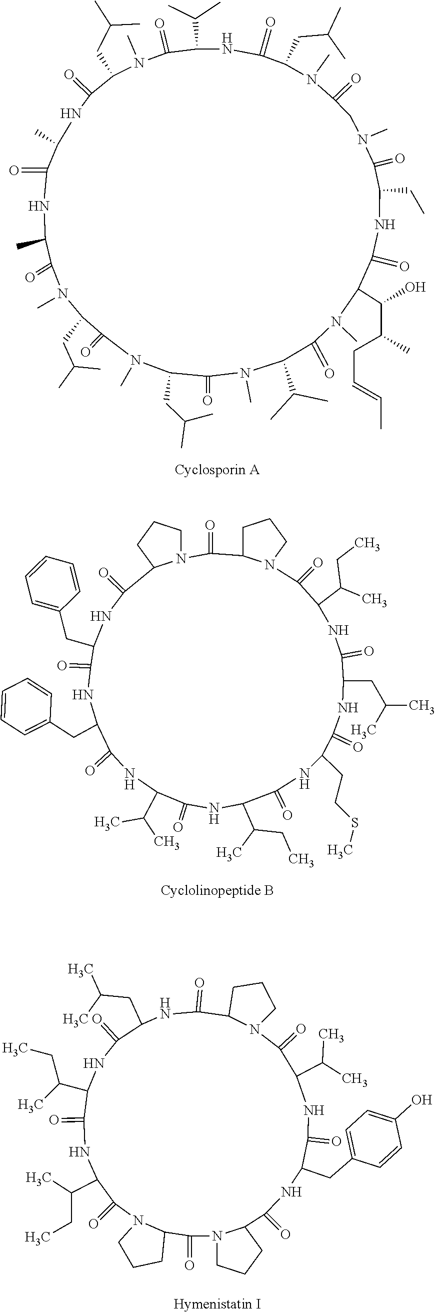

the cyclopeptide having a blood sugar regulating activity includes, but is not limited to, the following structures and similar structures thereof:

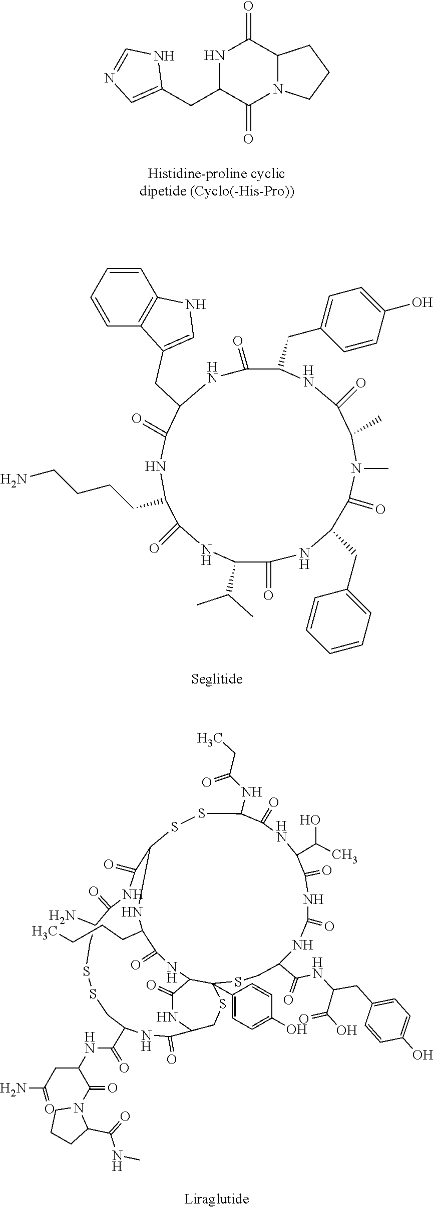

a cardiovascular and blood-related cyclopeptide includes, but is not limited to the following structures and similar structures thereof:

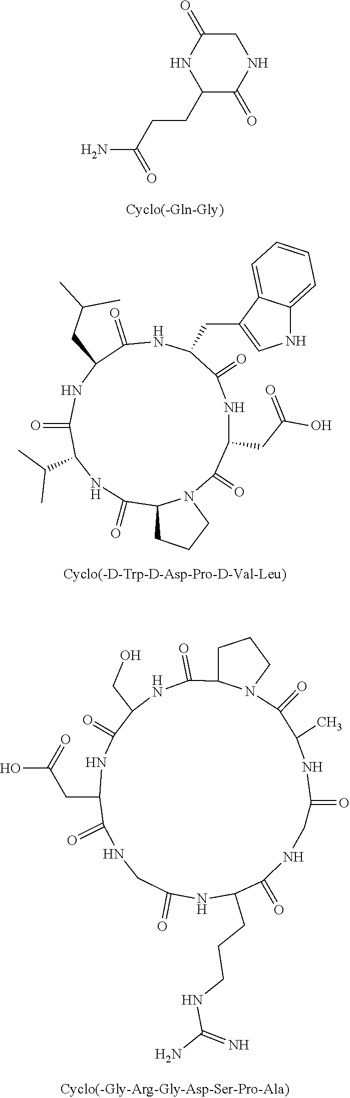 [SMILES: NC(=O)CCC1NC(=O)CNC1=O ; CC(C)C[C@@H]1NC(=O)[C@@H](C(C)C)NC(=O)[C@@H]2CCCN2C(=O)[C@@H](CC(=O)O)NC(=O)[C@@H](Cc2c[nH]c3ccccc23)NC1=O ; CC1NC(=O)CNC(=O)C(CCCNC(=N)N)NC(=O)CNC(=O)C(CC(=O)O)NC(=O)C(CO)NC(=O)C2CCCN2C1=O]

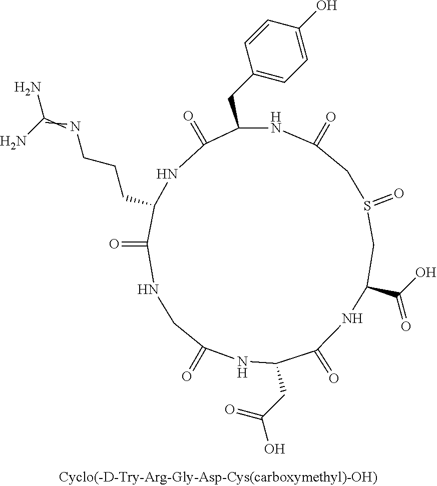 [SMILES: NC(N)=NCCC[C@@H]1NC(=O)[C@@H](Cc2ccc(O)cc2)NC(=O)CS(=O)C[C@@H](C(=O)O)NC(=O)[C@H](CC(=O)O)NC(=O)CNC1=O]

other active cyclopeptides include, but are not limited to the following structures and similar structures thereof:

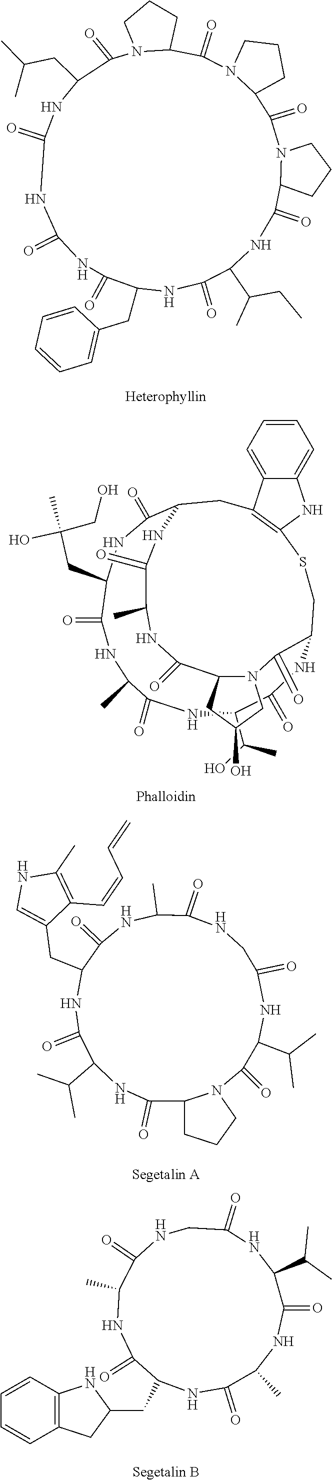

Preferably, a cyclopeptide derivative is a molecule, isomer or salt thereof with a similar structural skeleton to the above-mentioned cyclopeptide molecule.

Preferably, the cyclopeptide has poor water solubility, wherein the poor water solubility refers to that a maximum dissolved concentration of the cyclopeptide in pure water is less than or equal to 5 wt % at normal temperature and pressure.

In a second aspect: a cyclopeptide-based glass, wherein the glass is completely prepared from a cyclopeptide or a salt thereof; optionally, the glass further comprises other pharmaceutical adjuvants and/or active pharmaceutical ingredients.

A pharmaceutical composition glass consists of one or more of the following ingredients: cyclopeptides or cyclopeptide derivatives, other active pharmaceutical ingredients or pharmaceutically acceptable salts, pharmaceutically acceptable adjuvants thereof; and preferably, contains at least one active cyclopeptide;
the active pharmaceutical ingredients include one or a mixture of two or more of non-cyclopeptide drug molecules with antibacterial/antiviral, anti-tumor, blood sugar regulating, immunomodulatory, antipsychotic effects, etc. Preferably, an antibacterial/antiviral drug is penicillin, cephalexin, amikacin, norfloxacin, nitrofurantoin, metronidazole, amantadine, acyclovir, zidovudine, ribavirin; an anti-tumor drug is cisplatin, doxorubicin, vincristine, paclitaxel, docetaxel, gemcitabine, camptothecin, hydroxycamptothecin, irinotecan, etoposide, dexamethasone, fluorouracil, and cyclophosphamide; a blood sugar regulating drug is metformin, repaglinide, nateglinide, and insulin; an immunomodulatory drug is thymopentin, tripterygium glycoside, triptolide, tacrolimus, interferon, and coriolus versicolor polysaccharide; and an antipsychotic drug is chlorpromazine, haloperidol, risperidone, paliperidone, and ziprasidone;
the pharmaceutical adjuvants include fillers, wetting agents, binders, disintegrants, lubricants and others; preferably, the fillers are starch, lactose, and mannitol; the wetting agents/binders are sodium carboxymethyl cellulose and hydroxypropyl cellulose; the disintegrants are sodium carboxymethyl starch, low-substituted hydroxypropyl cellulose, cross-linked polyvinylpyrrolidone, and cross-linked sodium carboxymethyl cellulose; the lubricants are polyethylene glycol, magnesium stearate, and hydrogenated vegetable oil; and the others include sodium lauryl sulfate;
a mass proportion of the cyclopeptide ranges from 1% to 100%, preferably from 1% to 50%;
a mass proportion of other active pharmaceutical ingredients ranges from 1% to 20%, preferably from 1% to 10%; and
a mass proportion of the pharmaceutically acceptable adjuvants ranges from 0% to 50%, preferably from 0% to 5%, and further preferably 0%; that is, the cyclopeptide glass completely replaces traditional pharmaceutical adjuvants.

In a third aspect: a cyclopeptide-based glass may be used as a pharmaceutical adjuvant and may completely or partially replace a pharmaceutical adjuvant.

In a fourth aspect: a method for preparing a cyclopeptide glass drug or cyclopeptide glass adjuvant by converting a poorly soluble cyclopeptide into a glassy state mainly includes the following steps:
(1) performing full ball-milling treatment on one or more poorly soluble cyclopeptides to obtain a fully milled cyclopeptide raw material, wherein a temperature of a raw material during the ball-milling is controlled at 0° C. to 50° C., preferably 10° C. to 30° C.; and
(2) preparing a cyclopeptide glass drug or cyclopeptide glass adjuvant by a “heating-quenching” method which comprises heating the fully milled cyclopeptide raw material to a temperature near a melting point in an inert gas atmosphere, performing heat preservation for a period of time, and transferring to an annealing furnace for annealing treatment;
wherein,
a temperature of the heating is a temperature of a melting point temperature (Tₘ)±50 to 250 K, preferably 50 to 100 K higher than Tₘ;
the heat preservation is performed for 0 min to 30 h, preferably 15 to 30 min;
a temperature of the annealing treatment is a temperature of a glass transition temperature (T_{g})±50 to 150 K, preferably, 50 to 100 K lower than T_{g};
the annealing treatment is performed for 30 min to 2 h, preferably 30 min to 1 h; and
Tₘ and T_{g} are measured by thermogravimetric analysis and differential scanning calorimetry, and heating and cooling rates are 2 to 50 K min-1.

In a fifth aspect: a method for preparing a pharmaceutical composition glass comprising a poorly soluble cyclopeptide mainly includes the following steps:
(1) mixing a poorly soluble cyclopeptide with other active pharmaceutical ingredients, and performing ball-milling treatment on a resulting mixture, wherein a temperature of a raw material during the ball-milling is controlled at 0° C. to 50° C., preferably 10° C. to 30° C.;
(2) following the step (2) in the fourth aspect; and
(3) preparing the glass obtained in step (2) and pharmaceutically acceptable adjuvants into the pharmaceutical composition by means of one or a combination of two or more of tableting, wet granulation, fluidized-bed granulation, coating, spraying granulation, procedural pouring, and 3D printing.

Or, a preparation method mainly includes the following steps:
(1) mixing a poorly soluble cyclopeptide with other active pharmaceutical ingredients, and performing full ball-milling treatment on a resulting mixture to obtain a powder, wherein a temperature of a raw material during the ball-milling is controlled at 0° C. to 50° C., preferably 10° C. to 30° C.;
(2) heating the powder obtained in step (1) in an inert gas atmosphere, and performing heat preservation for a period of time, wherein a temperature of the heating is a melting point temperature (Tₘ)+50 to 250 K, preferably 50 to 100 K higher than Tₘ; the heat preservation is performed for 0 min to 30 h, preferably 15 to 30 min;
(3) completely dissolving other active pharmaceutical ingredients in a good solvent and co-solvent, wherein the good solvent is preferably ethanol, hexafluoroisopropanol, dichloromethane, carbon tetrachloride, acetone, or ethyl acetate, and the co-solvent is preferably one or a mixture of two or more of sodium benzoate, dimethylacetamide, urea, and Tween;
(4) mixing an active pharmaceutical ingredient solution obtained in step (3) and a molten cyclopeptide in step (1) evenly;
(5) placing a mixture obtained in step (4) at a temperature set in step (2) for rotary evaporation under a reduced pressure to remove a solvent;
(6) transferring a mixture obtained in step (5) to an annealing furnace for annealing treatment, wherein a temperature of the annealing treatment is a glass transition temperature (T_{g})±50 to 150 K, preferably 50 to 100 K lower than T_{g}, the annealing treatment time is performed for 30 min to 2 h, preferably 30 min to 1 h; and
(7) preparing the glass obtained in step (6) and pharmaceutically acceptable adjuvants into the pharmaceutical composition by means of one or a combination of two or more of tableting, wet granulation, fluidized-bed granulation, coating, spraying granulation, procedural pouring, and 3D printing.

Or, the preparation method mainly includes the following steps:
(1) completely dissolving a poorly soluble cyclopeptide and other active pharmaceutical ingredients together in a good solvent and co-solvent, wherein the good solvent is preferably ethanol, hexafluoroisopropanol, dichloromethane, carbon tetrachloride, acetone, or ethyl acetate, and the co-solvent is preferably one or a mixture of two or more of sodium benzoate, dimethylacetamide, urea, and Tween;
(2) heating a mixed solution obtained in step (1) in an inert gas atmosphere for rotary evaporation under a reduced pressure to remove a solvent, and then performing heat preservation treatment, wherein a temperature of the heating is a temperature of a melting point temperature (Tₘ)±50 to 250 K, preferably, 50 to 100 K higher than Tₘ; the heat preservation is performed for 0 min to 30 h, preferably 15 to 30 min;
(3) transferring a mixture obtained in step (2) to an annealing furnace for annealing treatment, wherein a temperature of the annealing treatment is a glass transition temperature (T_{g})±50 to 150 K, preferably 50 to 100 K lower than T_{g}, the annealing treatment is performed for 30 min to 2 h, preferably 30 min to 1 h; and
(4) preparing the glass obtained in step (3) and pharmaceutically acceptable adjuvants into pharmaceutical composition, by means of one or a combination of two or more of tableting, wet granulation, fluidized-bed granulation, coating, spraying granulation, procedural pouring, and 3D printing.

In a sixth aspect: the cyclopeptide glass drug, the cyclopeptide glass adjuvant, the cyclopeptide pharmaceutical composition glass and the preparation methods thereof according to the first aspect to the fifth aspect, wherein the dosage forms of glass drug may be an oral preparation, a patch, a subcutaneous embedding agent, a stent material and a microneedle device.

In a seventh aspect: the drug dosage form according to the sixth aspect, and the process involved comprises one or a combination of two or more of tableting, dry granulation, high shear wet granulation, fluidized-bed granulation, capsule canning, microcapsule embedding, coating, spray drying, spraying condensation, photoetching, procedural pouring, microneedle array, and 3D printing.

In an eighth aspect: the cyclopeptide glass, the cyclopeptide pharmaceutical composition glass, and the glassy dosage form of cyclopeptide drugs in the present invention have the following advantages and beneficial effects:
(1) being capable of exerting efficacy and functioning as a pharmaceutical adjuvant;
(2) being capable of effectively increasing a drug dissolution rate, improving the drug bioavailability, and reducing drug side effects;
(3) having good biocompatibility and biodegradability; and
(4) having high thermal/chemical stability.

In a ninth aspect: the cyclopeptide glass, the cyclopeptide pharmaceutical composition glass, and the glassy dosage form of cyclopeptide drugs in the present invention may be applied in the fields of drug delivery for resistance to tumors, resistance to viruses/bacteria, blood sugar control, immune modulation, neuromodulation, etc., especially to achieve sustained release of drugs.

The raw materials of the cyclopeptide glass of the present invention include cyclopeptide, especially include any one or a combination of two or more of cyclopeptides with biological/pharmacological activities or pharmaceutically acceptable derivatives, salts thereof; which generally have poor water stability, easy crystallization, and low bioavailability. The present invention creatively proposes a method for converting a poorly soluble cyclopeptide drug into a glassy state, including preparing a poorly soluble cyclopeptide into a glass, and preparing a poorly soluble cyclopeptide and other active pharmaceutical ingredients into a glass. The present invention provides a method for converting a poorly soluble drug into a glassy state to form a glassy dosage form of drugs, thereby realizing the sustained release of a poorly soluble active cyclopeptide and the drug. The resulting cyclopeptide glass has high biocompatibility, degradability and high thermal/chemical stability, and may be further processed into oral preparation, patches, subcutaneous embedding agents, stent materials and microneedle devices through spray drying, solid dispersion, additive manufacturing and other technologies. The obtained cyclopeptide glass can exert both efficacy and drug adjuvant function. Compared with crystals and traditional pharmaceutical dosage forms or adjuvants, the cyclopeptide glass can effectively increase a drug dissolution rate and improve drug bioavailability, and is widely applied in the fields of delivery and sustained release of drugs for resistance to tumors, resistance to viruses/bacteria, blood sugar control, immune regulation, and neuromodulation, etc.

### BRIEF DESCRIPTION OF THE DRAWINGS

FIG. 1 is a physical picture (a) of a CsA glass prepared in Example 1 at room temperature and a physical picture (b) of a CsA glass obtained through a pouring process, confirming the processability of a cyclopeptide glass.

FIG. 2 is a hydrogen nuclear magnetic spectrum of the CsA glass prepared in Example 1. Compared with a CsA raw material, peaks have not changed significantly, indicating that chemical components of cyclopeptide raw material molecules have not changed after heating, melting and annealing.

FIG. 3 is a differential scanning calorimetry (DSC) chart of the CsA glass prepared in Example 1, confirming its glassy structure, and its glass transition temperature (T_{g}=100.4 K).

FIG. 4 is an X-ray diffraction (XRD) pattern of the CsA glass prepared in Example 1, indicating that the CsA glass has an amorphous structure.

FIG. 5 shows stability test results of the CsA glass prepared in Example 1, confirming its high stability.

FIG. 6 is a drug sustained release curve of the CsA glass prepared in Example 1, proving the capability of achieving the sustained release of the drug.

FIG. 7 shows immune-related indicators of mice after oral administration of the CsA glass prepared in Example 1.

FIG. 8 is an XRD spectrum of a pharmaceutical composition glass based on CFP and a tumor chemotherapy drug PTX in Example 2, indicating an amorphous structure.

FIG. 9 shows the degradation of a pharmaceutical composition glass capsule prepared in Example 2 in artificial gastric juice (prepared according to a preparation method in Chinese Pharmacopoeia), confirming its degradable nature.

FIG. 10 is a drug-time curve of the pharmaceutical composition glass capsule prepared in Example 2 after intragastric administration in mice over time.

FIG. 11 shows a tumor inhibition curve of experimental mice after oral administration of the pharmaceutical composition glass capsule prepared in Example 2, confirming its anti-tumor effect.

FIG. 12 is a body weight change curve of experimental mice after oral administration of the pharmaceutical composition glass capsule prepared in Example 2, confirming high biological safety.

FIG. 13 shows the changes in organ coefficients of experimental mice after oral administration of the pharmaceutical composition glass capsule prepared in Example 2, confirming high biological safety.

FIG. 14 shows a picture and XRD data of a melting pharmaceutical composition glass of Example 3, confirming the transformation from crystals to a glassy state.

FIG. 15 shows biocompatibility test results of the pharmaceutical composition glass prepared in Example 3, confirming high biocompatibility.

FIG. 16 is a curve showing the inhibition of bacterial growth by a pharmaceutical composition glass coating prepared in Example 3.

FIG. 17 shows antibacterial effects of a pharmaceutical composition glass tablet prepared in Example 3 on Escherichia coli and Staphylococcus aureus.

FIG. 18 is a degradation curve over time after subcutaneous embedding of an embedding agent of the pharmaceutical composition glass prepared in Example 3.

FIG. 19 shows a control curve of a pharmaceutical composition glass tablet prepared in Example 4 on the blood sugar of spontaneous type II diabetic mice, confirming the capability of effectively controlling the blood sugar of mice.

FIG. 20 shows a change curve of body weights of mice over time after intragastric administration of the pharmaceutical composition glass tablet prepared in Example 4 to spontaneous type II diabetic mice, confirming the capability of effectively controlling the body weights of mice.

FIG. 21 shows the tolerance of oral administration with high-concentration glucose to spontaneous type II diabetic mice after 30 days of intragastric administration of the pharmaceutical composition glass tablet prepared in Example 4; wherein a glucose concentration is 2.5 g kg⁻¹. By measuring a blood glucose concentration in the plantar blood of mice and drawing a curve, it is confirmed that the mice in the experimental group have better glucose tolerance.

FIG. 22 is a DSC curve of a CPY—CFP-CLP cyclopeptide mixed glass prepared in Example 5. The glass transition temperatures of a single glass and a mixed glass are obtained, indicating that mixing a plurality of cyclopeptides can effectively suppress the crystallization tendency of a single cyclopeptide.

FIG. 23 is a release curve of CLP molecules from the cyclopeptide mixed glass prepared in Example 5 and CLP crystals, which shows that the cyclopeptide mixed glass can increase a dissolution rate of a CLP active cyclopeptide.

FIG. 24 is an XRD pattern of a pharmaceutical composition glass prepared in Example 5, which is amorphous.

FIG. 25 shows statistical data of autonomous activity abilities and anxiety-like behaviors of mice in an open field experiment. The results show that compared with normal mice, an activity distance in a central area of early socially isolated mice is significantly reduced, which, however, is significantly improved after treatment with glass microneedle patches.

FIG. 26 shows the number of times mice entered open and closed arms within 5 min and the residence time of the mice in the two arms in a maze experiment. The results show that compared with normal mice, the number of times the early socially isolated mice entered the open arm is significantly reduced, and the residence time of the mice in the open arm is shortened, which are significantly improved after treatment with glass microneedle patches.

### DETAILED DESCRIPTION

The technical solutions of the present invention are described in detail below through examples, but the protected content of the present invention is not limited thereto.

### Example 1

A method for preparing a cyclosporin A (CsA)-based cyclopeptide glass included the following steps:
(1) milling a CsA powder in a ball mill evenly, during which the ball mill was controlled at a constant temperature of 25° C., and then transferring to a crucible;
(2) placing the crucible filled with the CsA powder in step (1) in a heating equipment in an N₂ atmosphere;
(3) heating the equipment in step (2), heating the crucible from room temperature to 573.15 K at a heating rate of 10 K min⁻¹, and performing heat preservation at 573.15 K for 20 min;
(4) cooling the equipment in step (3), cooling the crucible to 273.15 K at a cooling rate of 10 K min⁻¹, performing heat preservation at 273.15 K for 30 minutes in an annealing furnace, and annealing the glass to obtain a CsA glass.

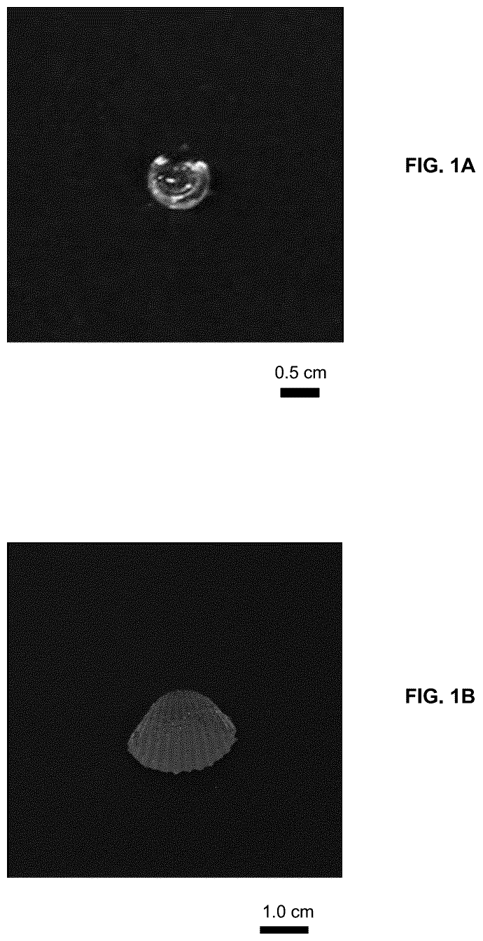

FIG. 1 is a physical picture (a) of the CsA glass prepared in Example 1 at room temperature and a physical picture (b) of a CsA glass obtained through a pouring process, confirming the processability of a cyclopeptide glass.

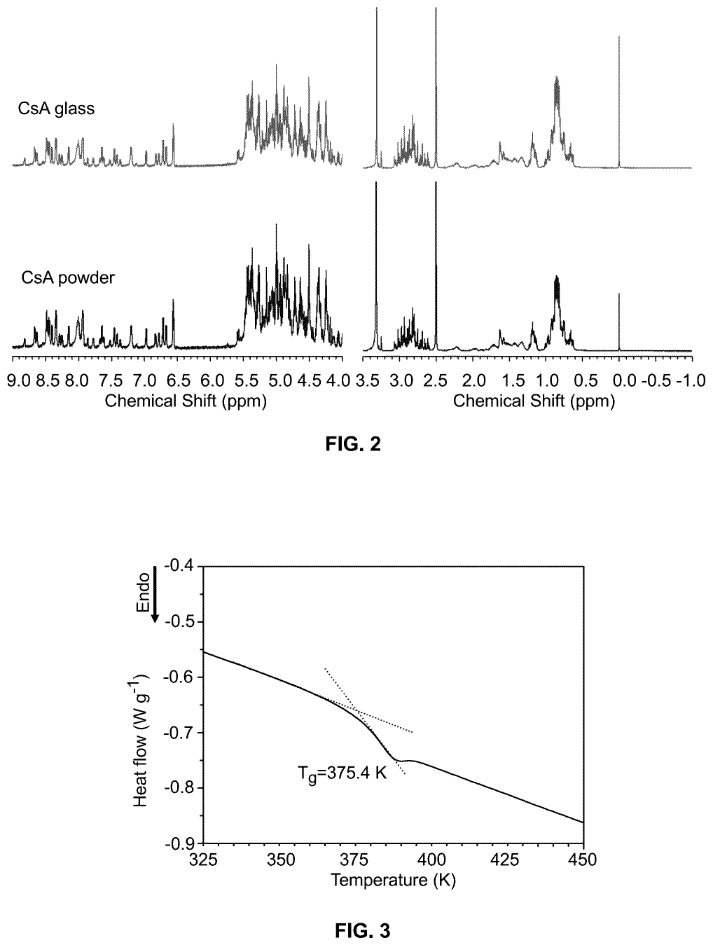

FIG. 2 shows a hydrogen nuclear magnetic spectrum of the CsA glass prepared in Example 1. Compared with a CsA raw material, peaks have not changed significantly, indicating that chemical components of cyclopeptide raw material molecules have not changed after heating, melting and annealing.

FIG. 3 is a DSC chart of the CsA glass prepared in Example 1, confirming its glassy structure, and its glass transition temperature (T_{g}=100.4 K).

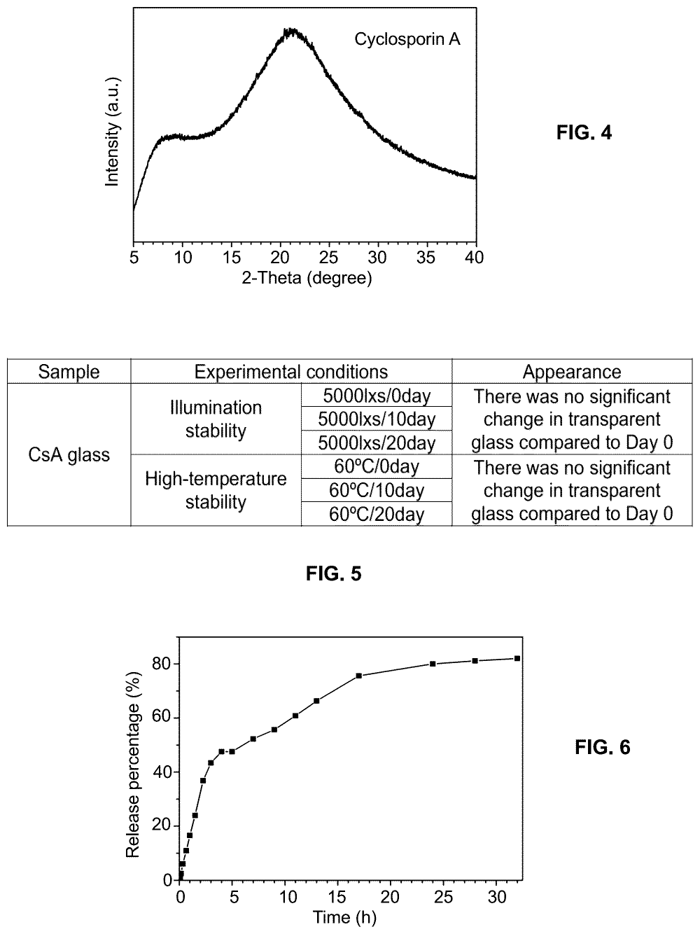

FIG. 4 is an X-ray diffraction (XRD) pattern of the CsA glass prepared in Example 1, indicating that the CsA glass has an amorphous structure.

FIG. 5 shows stability test results of the CsA glass prepared in Example 1, confirming its high stability.

FIG. 6 is a drug sustained release curve of the CsA glass prepared in Example 1, confirming the capability of achieving the sustained release of the drug. Specifically, an initial mass of the CsA glass was 20±1 mg, a sustained-release solvent was 10 mL phosphate buffer (0.01 M, pH=6.8), a stirring rate was 100 r min⁻¹, the dissolved CsA was tested through a dissolution tester and a high-performance liquid chromatograph and a drug sustained release curve was drawn.

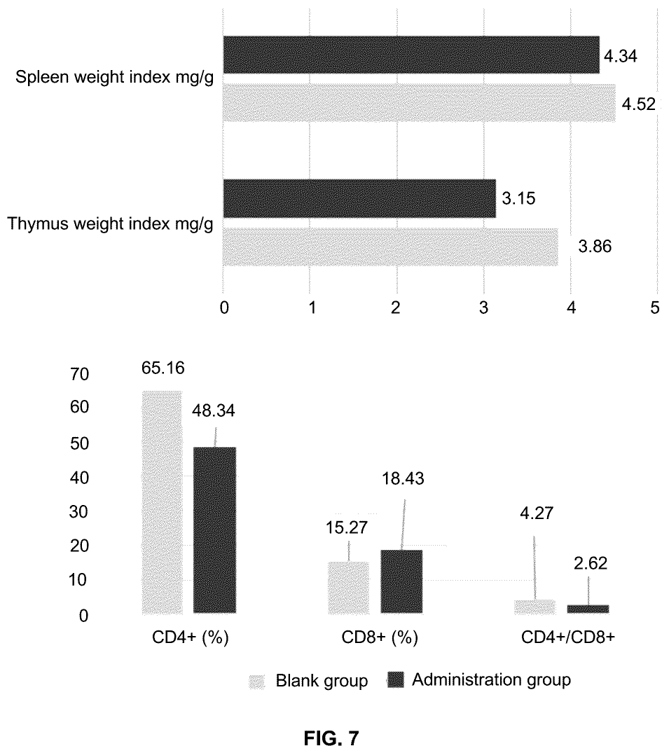

FIG. 7 shows immune-related indicators of mice after oral administration of the CsA glass prepared in Example 1. Specifically, healthy male Kunming rats (body weight of 20±1 g, 4-5 weeks old) were randomly divided into two groups, 8 rats in each group, and fed with a normal diet. Mice in an experimental group were intragastrically administered with the CsA glass every other day at a dose of 40 mg kg⁻¹ for 5 times. Mice in a control group were intragastrically administered with the same mass of normal saline every other day. The mice were euthanized the day after the last oral administration and the corresponding indicators were detected. The thymus and spleen of the mouse were dissected, and the surface blood was washed away with phosphate buffer saline (PBS). The water was absorbed to dryness by filter paper, and then weighed on an electronic balance. The weight of the organ (mg) was divided by a total weight (g) of the mouse to calculate an organ weight index. The peripheral blood of the mouse was taken, mixed with a CD3⁺(PE-Cy5) monoclonal antibody, a CD4⁺(PE) monoclonal antibody and a CD8⁺(APC) monoclonal antibody, the resulting mixture was shaken well, incubated in the dark, and analyzed by a flow cytometer after cell lysis and centrifugation.

### Example 2

A method for preparing a pharmaceutical composition glass based on phenylalanine-proline cyclic dipeptide (CFP) and paclitaxel (PTX) included the following steps:
(1) placing a CFP powder in a ball mill for milling and mixing evenly, during which a constant temperature of the ball mill was controlled at 37° C., and then transferring to a container;
(2) heating the container in step (1) from room temperature to 473.15 K at a heating rate of 20 K min⁻¹, and performing heat preservation at 473.15 K for 30 min;
(3) cooling the container in step (2) to a temperature of 333.15 K at a cooling rate of 10 K min⁻¹;
(4) milling the PTX powder in the ball mill evenly, during which a constant temperature of the ball mill was controlled at 37° C., then adding ethanol for completely dissolving, wherein a molar ratio of CFP to PTX was 25:1;
(5) adding a solution in step (4) dropwise into the container in step (3), placing the container in a reduced pressure rotary evaporator at a temperature maintaining at 333.15 K to remove a solvent;
(6) cooling the equipment in step (5), cooling the container to 315.15 K at a cooling rate of 10 K min⁻¹, performing heat preservation at 315.15 K for 30 min in an annealing furnace to obtain the pharmaceutical composition glass based on CFP and the tumor chemotherapy drug PTX; completely dissolving the above glass in HFIP organic solution, and quantifying a concentration of PTX through high performance liquid chromatography, wherein a drug loading capacity of PTX was calculated to be 3.5±0.2%; and
(7) preparing an oral dosage form of cyclopeptide glass through a preparation process of spray drying and capsule infusion.

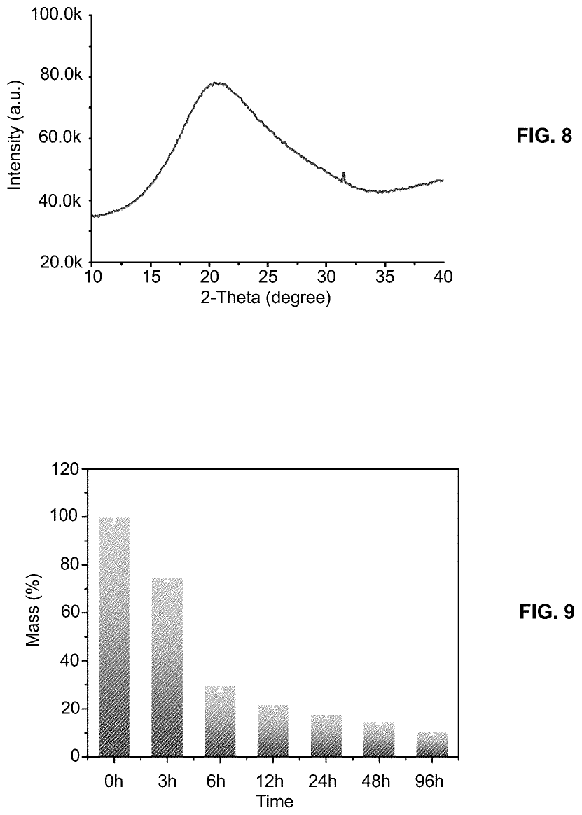

FIG. 8 is an XRD spectrum of the pharmaceutical composition glass based on CFP and the tumor chemotherapy drug PTX in Example 2, proving that the pharmaceutical composition of the present invention has an amorphous structure.

FIG. 9 shows the degradation of a pharmaceutical composition glass capsule prepared in Example 2 in artificial gastric juice (following the preparation method of the Chinese Pharmacopoeia), confirming its degradable nature. The preparation steps of the pharmaceutical composition glass capsule included milling the pharmaceutical composition glass into a powder through a homogenizer, and filling the above powder into a gelatin capsule shell.

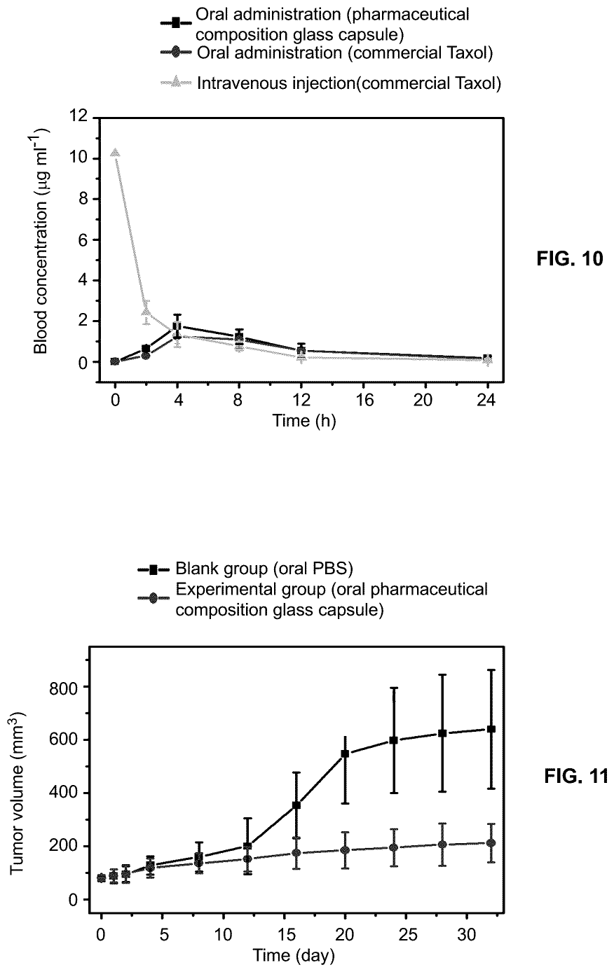

FIG. 10 is a drug-time curve of the pharmaceutical composition glass capsule prepared in Example 2 after intragastric administration in mice over time. Specifically, the mice were administered with a dose of 8 mg kg⁻¹. After intragastric administration, the peripheral blood of the mice was collected over time by orbital blood collection, followed by cell lysis and solvent dissolution. The filtered solution was detected with a high-performance liquid chromatograph. The PTX content in the blood was calculated, and the drug-time curve was drawn.

FIG. 11 shows a tumor inhibition curve of experimental mice after oral administration of the pharmaceutical composition glass capsule prepared in Example 2, confirming its anti-tumor effect. Specifically, healthy BALB/c nude mice (body weight 15±1 g, 4-6 weeks old) were randomly divided into two groups, with 8 mice in each group. 4T1 cells were subcutaneously inoculated into the axilla to establish a 4T1 mouse transplanted tumor model of breast cancer. After the mouse tumors grew to a volume of 80±10 mm³, the mice were treated with drugs. The mice in the experimental group were intragastrically administered with drug every other day at a dosage of 8 mg kg⁻¹ for a total of 5 times. The mice in the control group were intragastrically administered with the same mass of PBS every other day. The changes in mouse tumor volume were measure by a vernier caliper and record every day. The mouse tumor volume was calculated according to a formula, V=ab²/2, where a was a long side distance of the tumor, and b was a short side distance of the tumor.

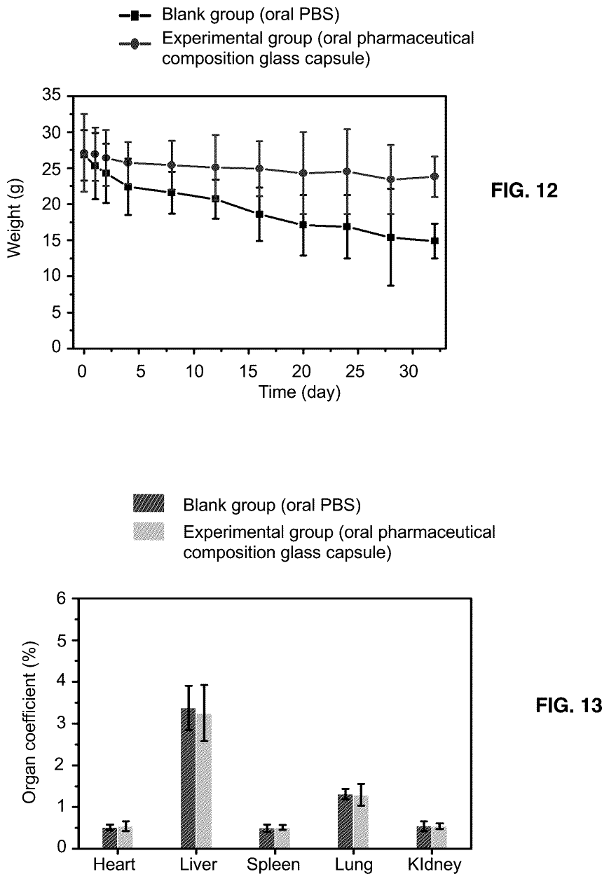

FIG. 12 is a body weight change curve of experimental mice after oral administration of the pharmaceutical composition glass capsule prepared in Example 2, confirming high biological safety.

FIG. 13 shows the changes in organ coefficients of experimental mice after oral administration of the pharmaceutical composition glass capsule prepared in Example 2, confirming high biological safety. Specifically, on the 35^{th} day, the important organs of the mice (including the heart, liver, spleen, lungs, and kidneys) were dissected, and the surface blood was washed away with PBS. The water was absorbed to dryness by filter paper, and then the organs were weighed on an electronic balance. The organ coefficient was calculated by dividing the weight (mg) of the organ by a total weight (g) of the mouse.

### Example 3

A method for preparing a pharmaceutical composition glass based on vancomycin, gramicidin S and penicillin included the following steps:
(1) milling and mixing evenly the vancomycin, gramicidin S and penicillin in a ball mill, during which a constant temperature of the ball mill was controlled at 37° C., and then transferring to a container, wherein a mass ratio of the three components was 2:2:1;
(2) adding a mixed organic solvent of hexafluoroisopropanol and ethanol to the mixed powder in step (1), and stirring until completely dissolved;
(3) placing a mixed solution obtained in step (2) into a reduced pressure rotary evaporator, heating from room temperature to 443.15 K at a heating rate of 50 K min⁻¹, and performing heat preservation at 443.15 K for 30 minutes to remove an organic solvent;
(4) cooling the equipment in step (3) to a temperature of 243.15 K at a cooling rate of 10 K min⁻¹, and performing heat preservation at 243.15 K for 20 min;
(5) adding a certain mass of pharmaceutically acceptable adjuvants to a product obtained in step (4), including 1.5 parts of starch, 1 part of sodium carboxymethylcellulose, 2.5 parts of polyethylene glycol and 6.5 parts of hydrogenated vegetable oil, wherein a total mass proportion of the adjuvants was 92.5%; and
(6) preparing an embedding agent for the pharmaceutical composition glass by homogenizing a pharmaceutical composition obtained in step (5) in combination with tableting and vacuum spray drying techniques.

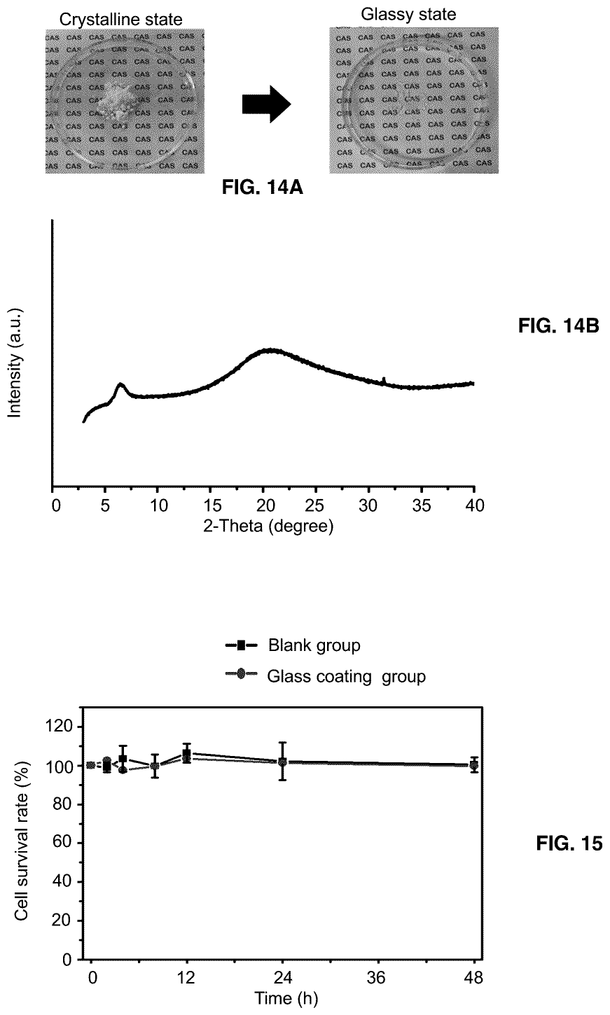

FIG. 14 shows a picture and XRD data of the melting pharmaceutical composition glass of Example 3, confirming the transformation from crystals to a glassy state.

FIG. 15 shows biocompatibility test results of the pharmaceutical composition glass prepared in Example 3, confirming high biocompatibility. Specifically, the above-mentioned glass was processed by a coating machine into the coating of cell culture dishes with a diameter of 60 mm and a thickness of 0.2 mm, which was used for cell culture (fibroblast 3T3). After a total of 48 hours of incubation, a survival rate of the cells was test by an MTT method.

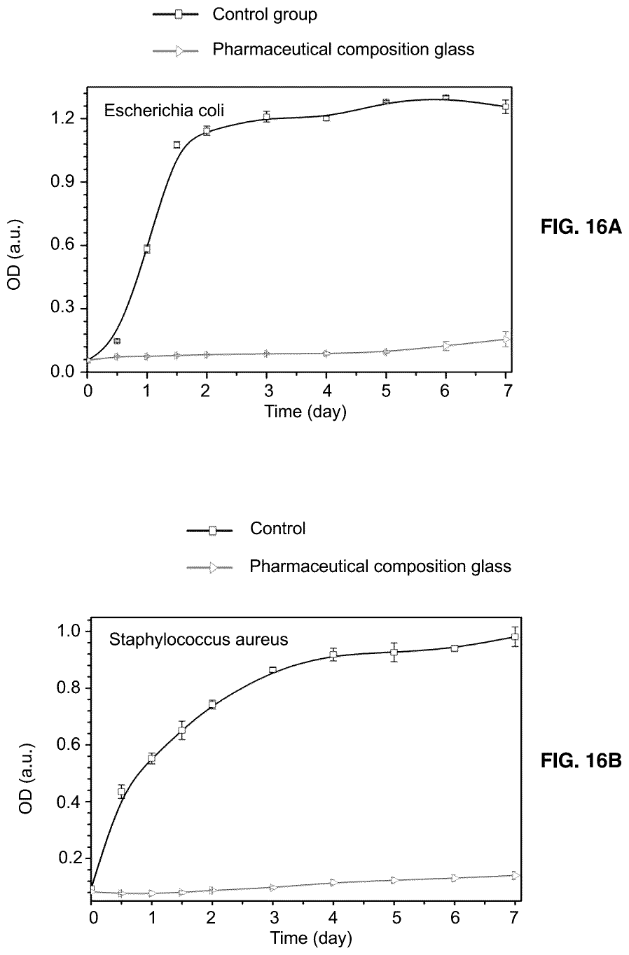

FIG. 16 is a curve showing the inhibition of bacterial growth by the pharmaceutical composition glass coating prepared in Example 3. Specifically, the above-mentioned pharmaceutical composition glass was processed into a tablet of 1 cm*1 cm*0.1 cm, and was attached to the center of a bacterial culture dish. The total drug concentration was calculated to be 10 μg/dish. Escherichia coli and Staphylococcus aureus were mixed evenly in a liquid culture medium, added dropwise into the bacterial culture dish, shaken and incubated at 37° C., and the bacterial concentration in the bacterial culture dish was measured over time by an OD value method.

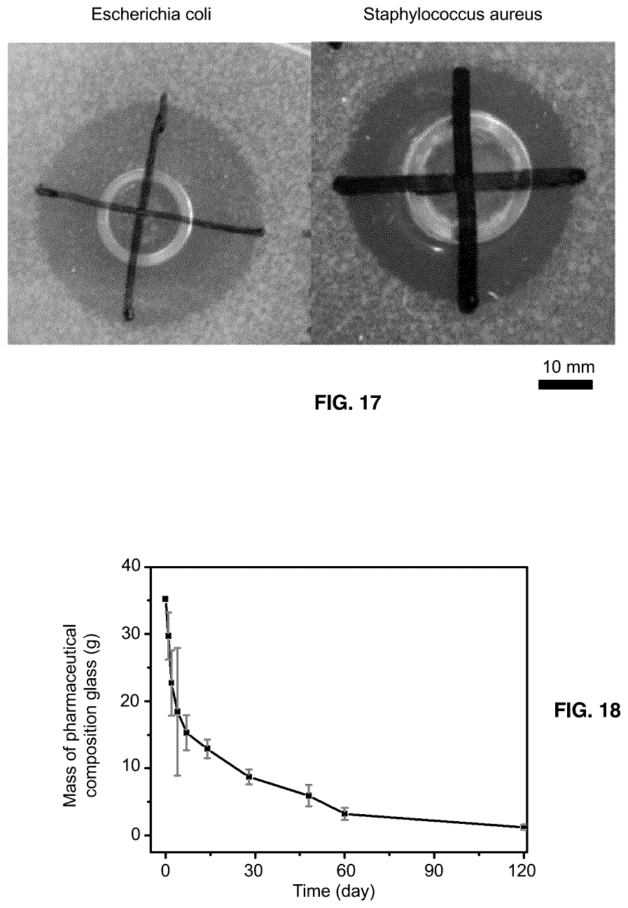

FIG. 17 shows antibacterial effects of the pharmaceutical composition glass tablet prepared in Example 3 on Escherichia coli and Staphylococcus aureus. Specifically, the above-mentioned pharmaceutical composition glass was processed into a tablet with a diameter of 5 mm and a thickness of 0.2 mm. An Oxford cup (a diameter of 6 mm) was placed on an agar petri dish. The tablet was placed in the Oxford cup and closely adhered to agar. Escherichia coli and Staphylococcus aureus were mixed with PBS evenly, the resulting mixture was coated on a dish, and placed in a 37° C. incubator for culture. After 24 h, whether an inhibition ring was produced or not was observed to determine an antibacterial effect of the pharmaceutical composition glass.

FIG. 18 is a degradation curve over time after subcutaneous embedding of the embedding agent for the pharmaceutical composition glass prepared in Example 3. Specifically, healthy male Kunming rats (body weight of 20±1 g, 4-5 weeks old) were randomly divided into two groups, with 15 rats in each group. A 1 cm window was surgically created on the back of the mice in the experimental group respectively, 30±2.5 g of the pharmaceutical composition glass was buried under the skin, and then the skin was sutured. Any one of mice was taken over time, the pharmaceutical composition glass was taken out, the remaining mass was weighed, and a degradation curve was drawn.

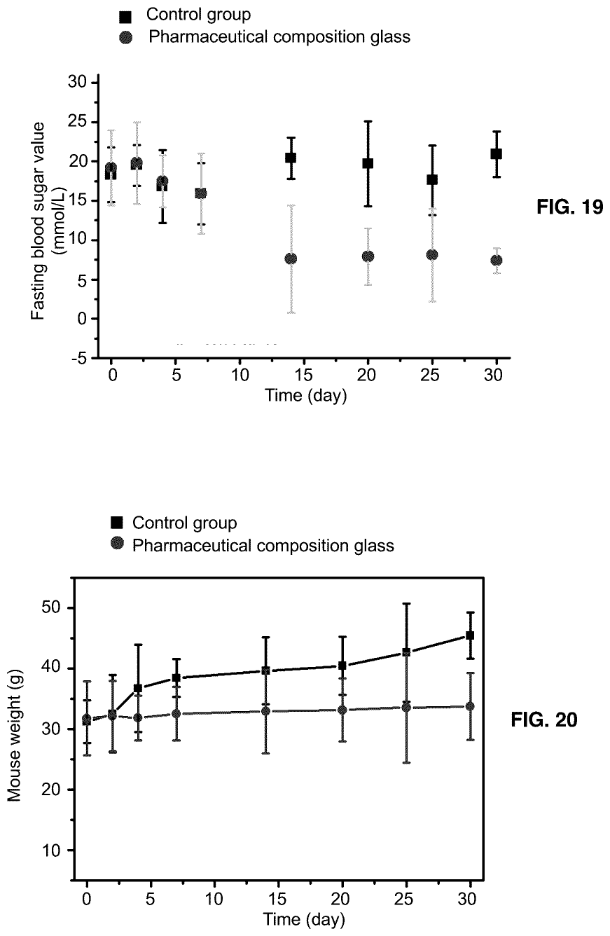

FIG. 19 shows a control curve of the pharmaceutical composition glass tablet prepared in Example 4 on the blood sugar of spontaneous type II diabetic mice, confirming the capability of effectively controlling the blood sugar of mice. Specifically, the ordered spontaneous type II diabetes model mice (male, 8 weeks old) were divided into two groups, with 6 mice in each group. The mice in the experimental group were intragastrically administered with the pharmaceutical composition glass every day at a dosage of 4 mg kg⁻¹, while the mice in the control group were intragastrically administered with an equal mass of normal saline. A test period was 30 days. The tail blood of the mice was taken every day, and the fasting blood glucose value of the mice was measured with a blood glucose tester. The curve is drawn by taking time as the abscissa and fasting blood glucose values of experimental mice as the ordinate.

FIG. 20 shows a change curve of body weights of mice over time after intragastric administration of the pharmaceutical composition glass tablet prepared in Example 4 to spontaneous type II diabetic mice, confirming the capability of effectively controlling the body weights of mice.

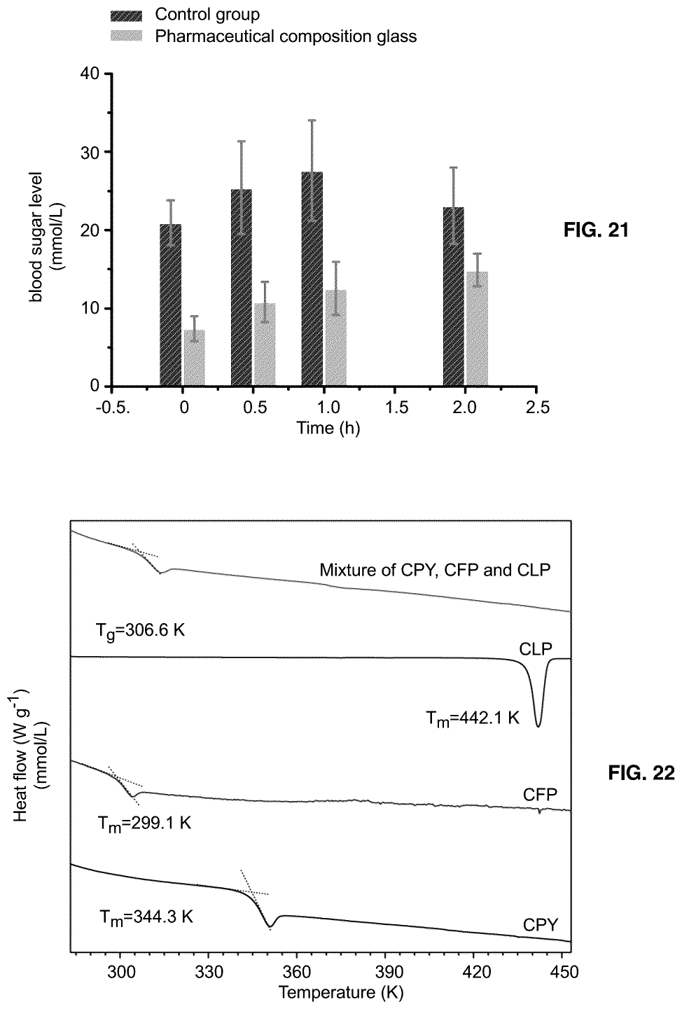

FIG. 21 shows the tolerance of oral administration with high-concentration glucose to spontaneous type II diabetic mice after 30 days of intragastric administration of the pharmaceutical composition glass tablet prepared in Example 4, wherein a glucose concentration was 2.5 g kg⁻¹. By measuring a blood glucose concentration in the plantar blood of mice and drawing a curve, it was confirmed that the mice in the experimental group had better glucose tolerance.

FIG. 22 is a DSC curve of a CPY—CFP-CLP cyclopeptide mixed glass prepared in Example 5. The glass transition temperatures of a single glass and a mixed glass are obtained, indicating that mixing a plurality of cyclopeptides can effectively suppress the crystallization tendency of a single cyclopeptide.

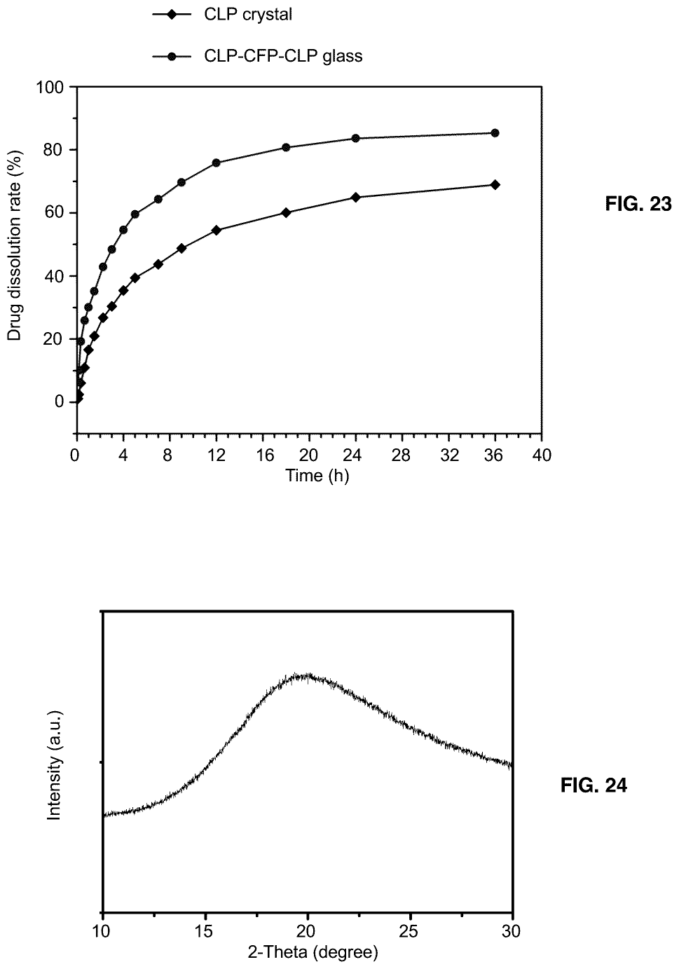

FIG. 23 is a release curve of CLP molecules from the cyclopeptide mixed glass prepared in Example 5 and CLP crystals, which shows that the cyclopeptide mixed glass can increase a dissolution rate of a CLP active cyclopeptide. Specifically, an initial mass of the cyclopeptide mixed glass was 15±1 mg, a sustained-release solvent was 10 mL PBS (0.01 M, pH=6.8), a stirring rate was 100 r min⁻¹. The dissolved CLP was detected through a dissolution tester and a high-performance liquid chromatograph, and a sustained release curve of the cyclopeptide mixed glass was drawn. As a control, CLP crystals of equal mass were weighed, added in the above sustained-release solution, stirred and incubated, and the concentration of CLP in the sustained-release solution was detected.

FIG. 24 is an XRD pattern of the pharmaceutical composition glass prepared in Example 6, which is amorphous.

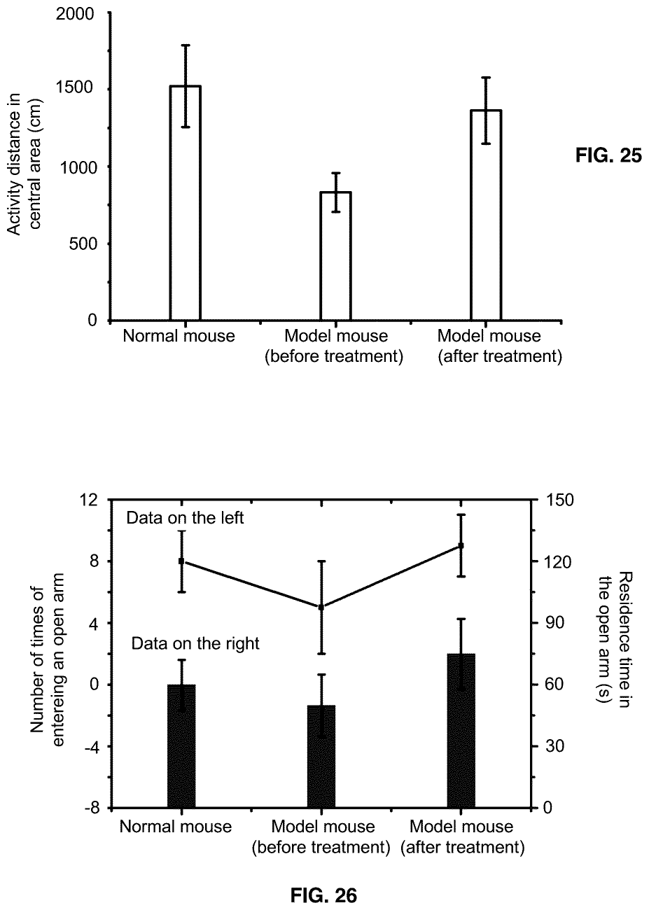

FIG. 25 shows statistical data of autonomous activity abilities and anxiety-like behaviors of mice when the pharmaceutical composition glass prepared in Example 6 is applied in an animal open field experiment. The results show that compared with normal mice, an activity distance in a central area of early socially isolated mice is significantly reduced, which is significantly improved after treatment with glass microneedle patches. Specifically, C57BL/6 early socially isolated mice (8 weeks old, male) were evenly divided into 2 groups, with 6 mice in each group. The mice in one group received no special treatment, and the mice in the other group had glass microneedle patches attached to them after hair removal on their backs. Six C57BL/6 normal mice were group-raised as a blank control group. According to the standards, a black mine device (45 cm*45 cm*45 cm) was prepared, the mice were placed in the mine, and the autonomous activity behaviors of the mice were tracked and recorded. The ordinate was statistical values of the activity distance in the center area of mice moving in a mine field for 1 h.

FIG. 26 shows the number of times the mice entered open and closed arms within 5 min and the residence time of the mice in the two arms when the pharmaceutical composition glass prepared in Example 6 is applied to a mouse maze experiment. The results show that compared with normal mice, the number of times the early socially isolated mice entered the open arm is significantly reduced, and the residence time of the mice in the open arm is shortened, which are significantly improved after treatment with glass microneedle patches. Specifically, according to the standards, a maze device with an arm width of 5 cm, an arm length of 35 cm, a closed arm height of 15 cm, a central platform of 5 cm*5 cm was prepared, and a maze height of about 40 to 55 cm from the ground. The mouse was placed in the center of the platform, the number of times the mouse entered the open and closed arms and the residence time of the mouse in the two arms was recorded, and anxiety indicators were evaluated.

### Example 4

A method for preparing a pharmaceutical composition glass based on histidine-proline cyclic dipeptide and seglitide included the following steps:
(1) placing the histidine-proline cyclic dipeptide and seglitide in a ball mill for milling and mixing evenly, during which a constant temperature of the ball mill was controlled at 25° C., and then transferring to a container, wherein a mass ratio of the histidine-proline cyclic dipeptide to the seglitide was 10:1;
(2) adding a methylene chloride solvent to a mixed powder in step (1) and stirring until completely dissolved;
(3) placing a mixed solution obtained in step (2) into a reduced pressure rotary evaporator, wherein the reduced pressure rotary evaporator was heated from room temperature to 535.15 K at a heating rate of 10 K min⁻¹, and performed heat preservation at 535.15 K for 30 min, to remove an organic solvent;
(4) cooling the equipment in step (3) to a temperature of 283.15 K at a cooling rate of 10 K min⁻¹, and performing heat preservation at 283.15 K for 1 h to obtain the pharmaceutical composition glass based on histidine-proline cyclic dipeptide and seglitide; and
(5) homogenizing the pharmaceutical composition glass obtained in step (5), and preparing into tablets for oral administration for blood sugar control through tableting and procedural pouring.

### Example 5

A method for preparing a mixed glass based on proline-tyrosine cyclic dipeptide (CPY), phenylalanine-proline cyclic dipeptide (CFP) and leucine-proline cyclic dipeptide (CLP) included the following steps:
(1) weighing CPY, CFP, and CLP powders in equal molar ratio and dissolving them in hexafluoroisopropanol to form a single cyclopeptide solution;
(2) placing a mixed solution obtained in step (1) into a reduced pressure rotary evaporator, wherein the reduced pressure rotary evaporator was heated from room temperature to 473.15 K at a heating rate of 10 K min⁻¹, and performed heat preservation at 473.15 K for 30 min, to remove an organic solvent; and
(3) cooling the equipment in step (2) to a temperature of 243.15 K at a cooling rate of 10 K min⁻¹, and performing heat preservation at 243.15 K for 30 min to obtain the CPY—CFP-CLP cyclopeptide mixed glass.

### Example 6

A method for preparing a pharmaceutical composition glass based on tryptophan-tryptophan cyclic dipeptide (CWW) and risperidone included the following steps:
(1) weighing CWW and risperidone powder in an equal molar ratio, and dissolving them in hexafluoroisopropanol and methanol respectively to form monodisperse solutions;
(2) placing a mixed solution obtained in step (1) into a reduced pressure rotary evaporator, wherein the reduced pressure rotary evaporator was heated from room temperature to 573.15 K at a heating rate of 10 K min⁻¹, and performed heat preservation at 573.15 K for 30 min, to remove an organic solvent;
(3) cooling the equipment in step (2) to a temperature of 283.15 K at a cooling rate of 50 K min⁻¹, and performing heat preservation at this temperature for 30 min to obtain the pharmaceutical composition glass based on CWW and risperidone; and
(4) processing the pharmaceutical composition glass obtained in step (3) into a microneedle patch with a patch size of 0.8*0.8 cm for transdermal drug delivery based on a 3D printing process and a pouring process. An early socially isolated mouse model of C57BL/6 was established, treated with the subcutaneous microneedle patch with a dose of risperidone of 5 mg kg⁻¹, and evaluated schizophrenia-like behaviors of the mice after 14 days after 14 days.

The invention claimed is:

1: A cyclopeptide-based glass, wherein the cyclopeptide is a cyclopeptide having a structural formula 1 or a salt thereof, and the cyclopeptide is one or a combination of two or more of cyclopeptides, 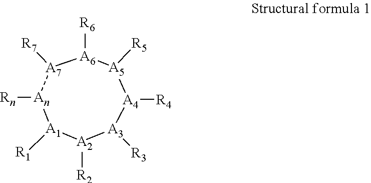
wherein A₁-Aₙ are independently selected from the group consisting of:
glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, selenocysteine and pyrrolysine;
R₁-Rₙ are independently selected from the group consisting of H and other modifiable groups, and the modifiable groups are independently selected from the group consisting of methyl, alkyl, phosphoric acid, acetyl, formyl, fatty acid, benzoyl, amide, ester, 9-fluorenylmethyloxycarbonyl, and tert-butoxycarbonyl;
n≥2, A₁-Aₙ are linked by amino acid condensation;
the cyclopeptide has an antibacterial/antiviral activity, an anti-tumor activity, a blood sugar regulating activity, an immunomodulatory activity, a cardiovascular and blood-related activity and other activities.

2: The cyclopeptide-based glass according to claim 1, wherein
the cyclopeptide having an antibacterial/antiviral activity is one or a combination of the following cyclopeptides: 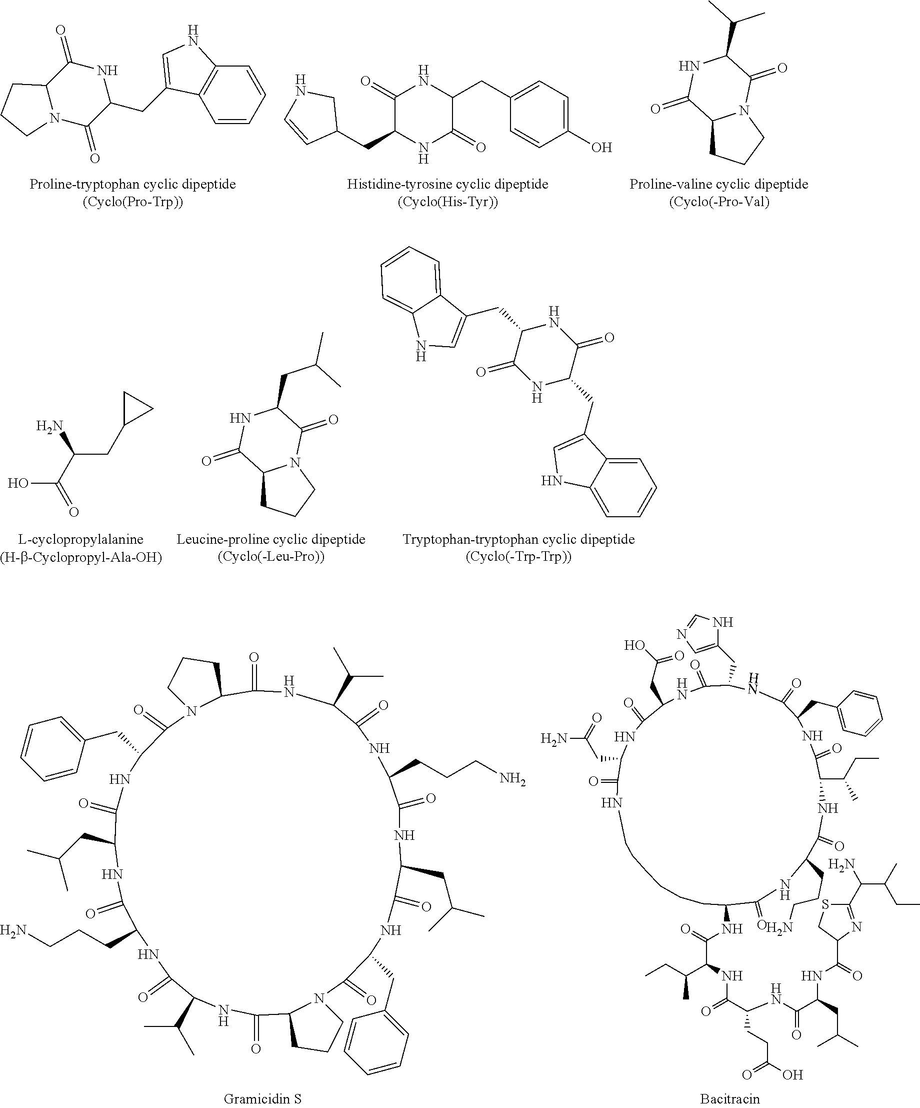 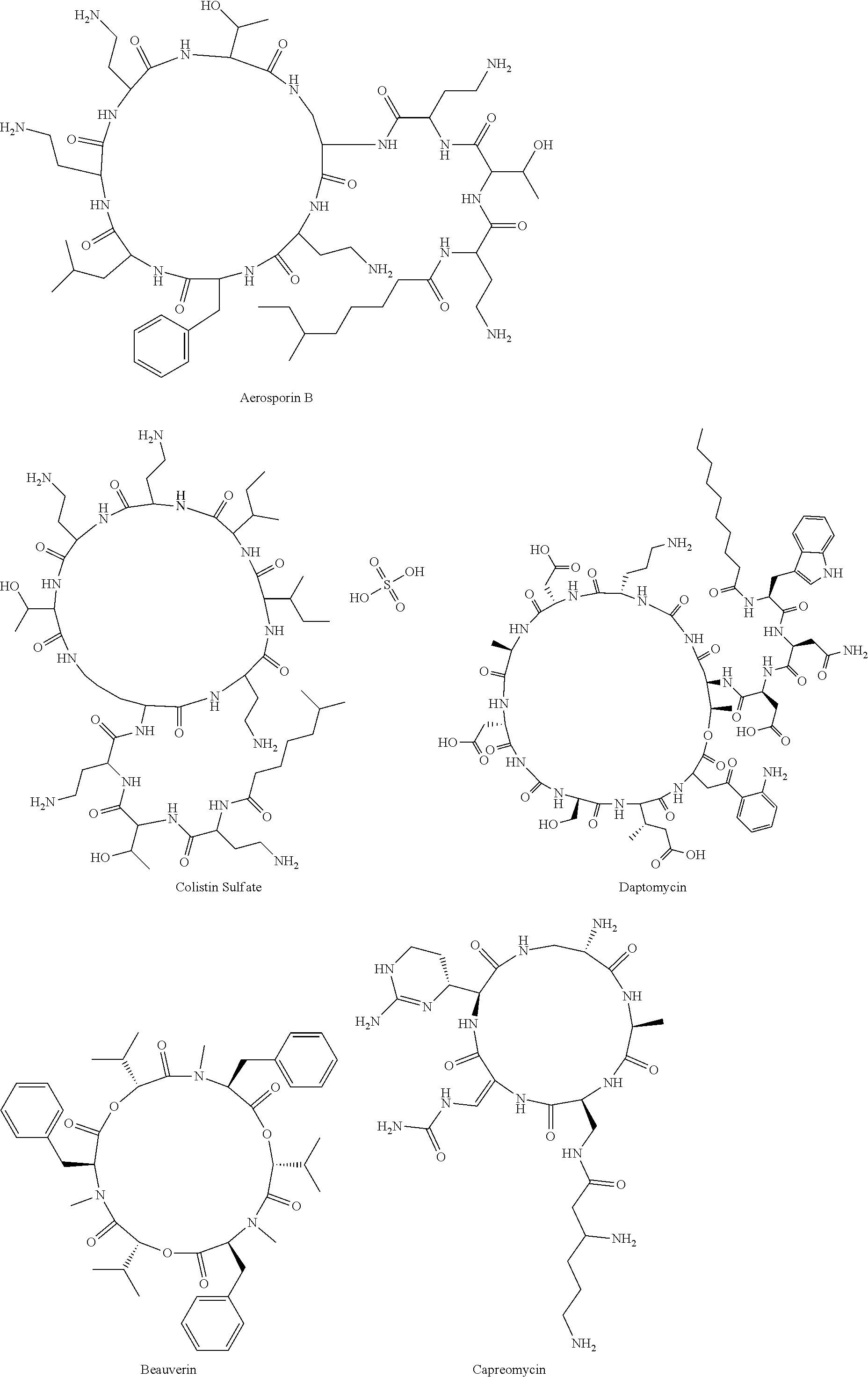 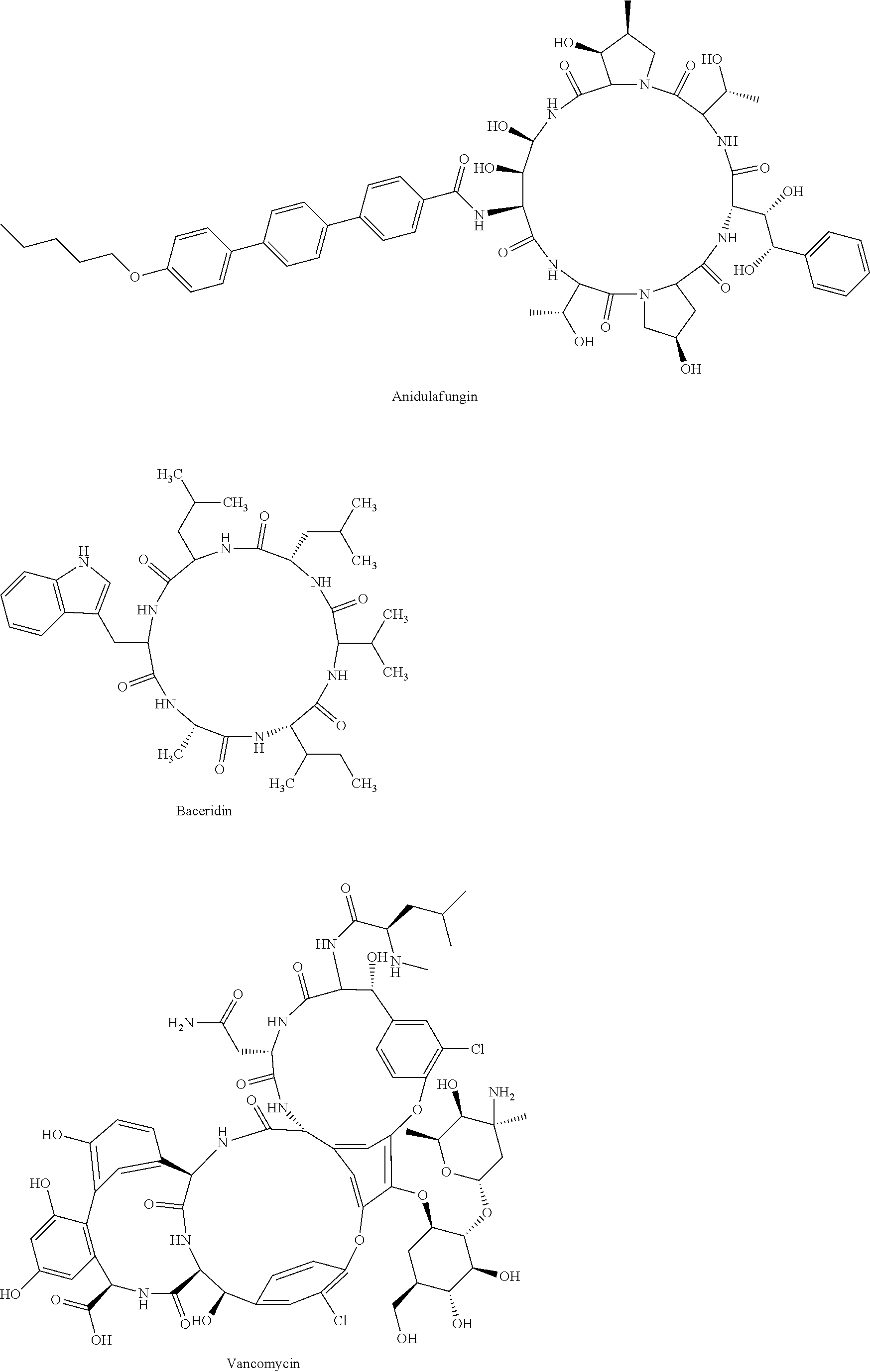 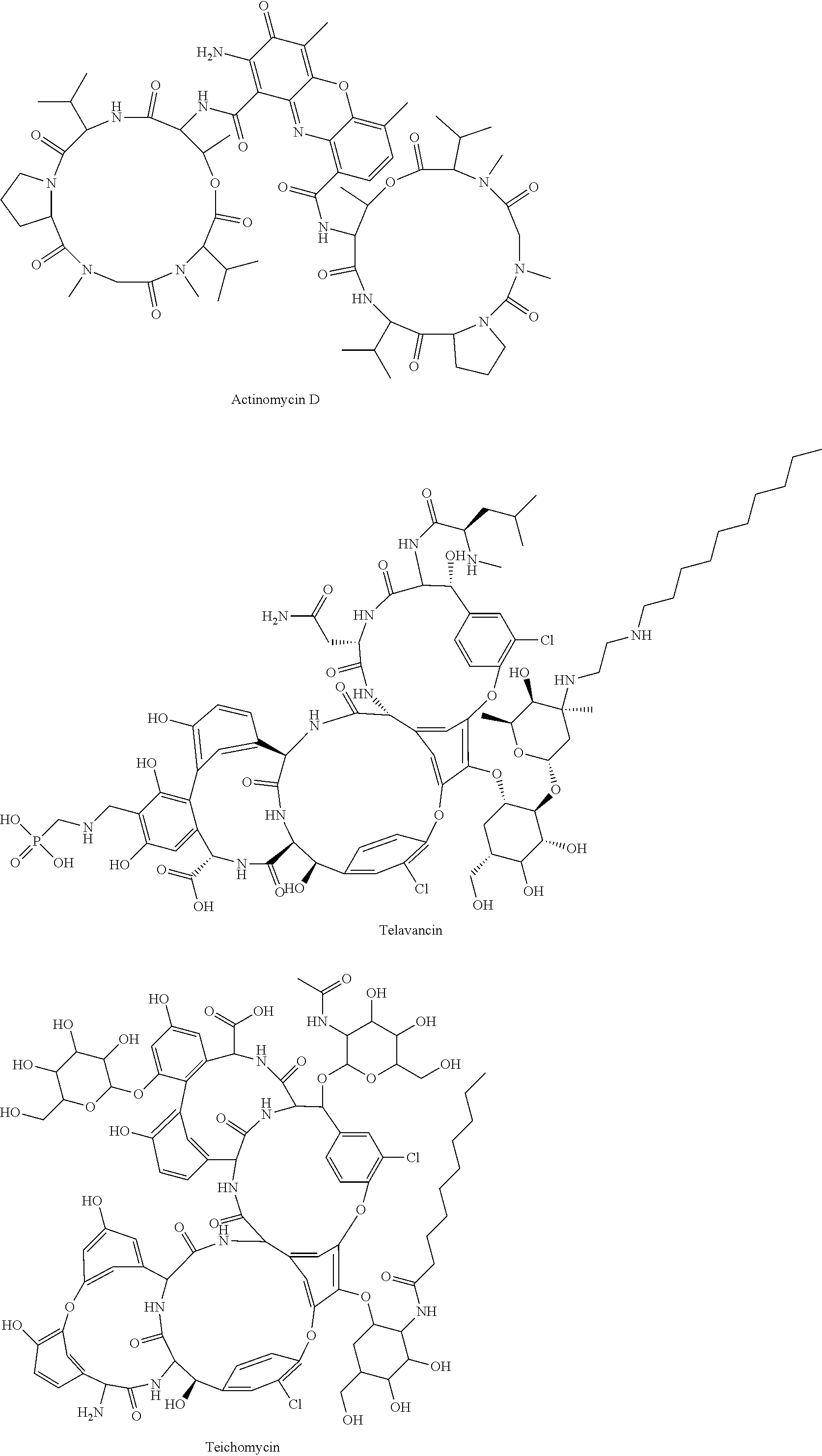 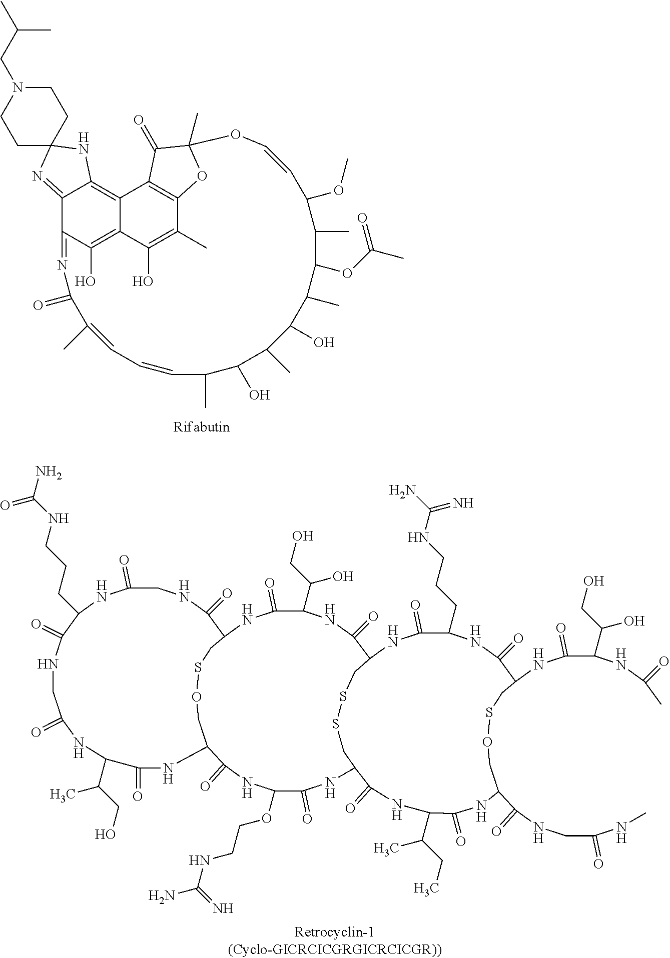 [SMILES: COC1/C=C\OC2(C)Oc3c(C)c(O)c4c(O)/c(c5c(c4c3C2=O)NC2(CCN(CC(C)C)CC2)N=5)=N\C(=O)/C(C)=C\C=C/C(C)C(O)C(C)C(O)C(C)C(OC(C)=O)C1C ; CCC(C)C1NC(=O)C2CSSCC(NC(=O)C(CCCNC(=N)N)NC(=O)C(NC(=O)C(NC(C)=O)C(O)CO)CSOCC(C(=O)NCC(=O)NC)NC1=O)C(=O)NC(C(O)CO)C(=O)NC1CSOCC(NC(=O)C(C(C)CO)NC(=O)CNC(=O)C(CCCNC(N)=O)NC(=O)CNC1=O)C(=O)NC(OCCNC(=N)N)C(=O)N2]
the cyclopeptide having an anti-tumor activity is one or a combination of the following cyclopeptides: 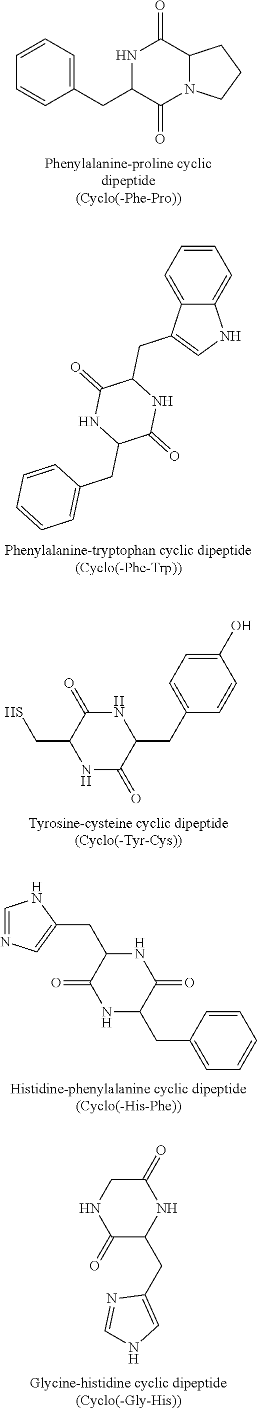 [SMILES: O=C1NC(Cc2ccccc2)C(=O)N2CCCC12 ; O=C1NC(Cc2c[nH]c3ccccc23)C(=O)NC1Cc1ccccc1 ; O=C1NC(Cc2ccc(O)cc2)C(=O)NC1CS ; O=C1NC(Cc2cnc[nH]2)C(=O)NC1Cc1ccccc1 ; O=C1CNC(=O)C(Cc2c[nH]cn2)N1] 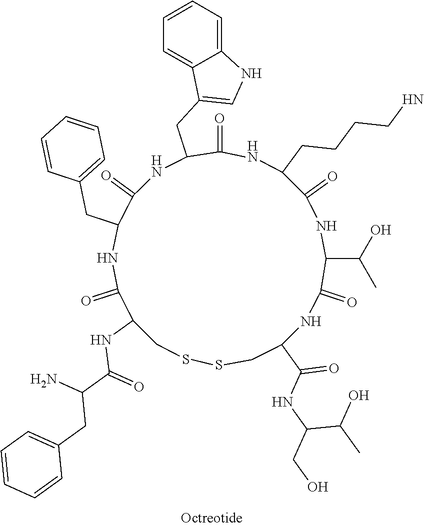 [SMILES: CC(O)C(CO)NC(=O)C1CSSCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1] 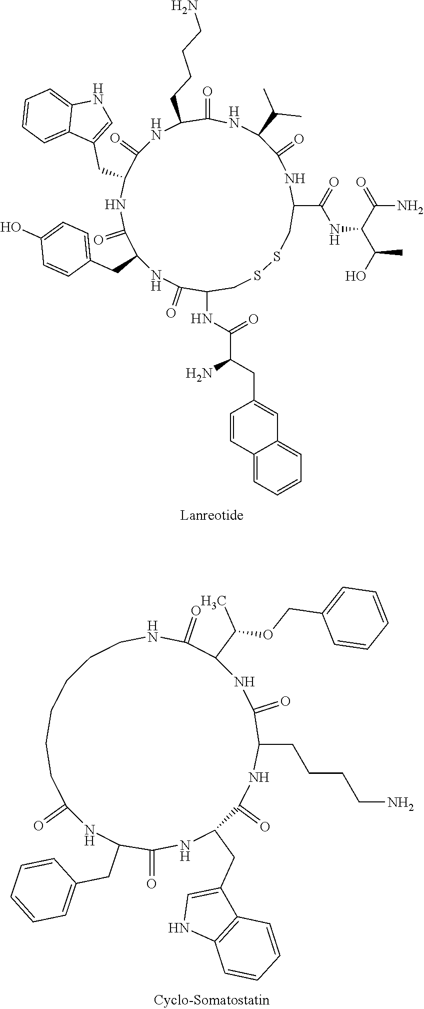 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)C(NC(=O)[C@H](N)Cc2ccc3ccccc3c2)CSSCC(C(=O)N[C@H](C(N)=O)[C@@H](C)O)NC1=O ; C[C@H](OCc1ccccc1)C1NC(=O)C(CCCCN)NC(=O)[C@H](Cc2c[nH]c3ccccc23)NC(=O)C(Cc2ccccc2)NC(=O)CCCCCCNC1=O] 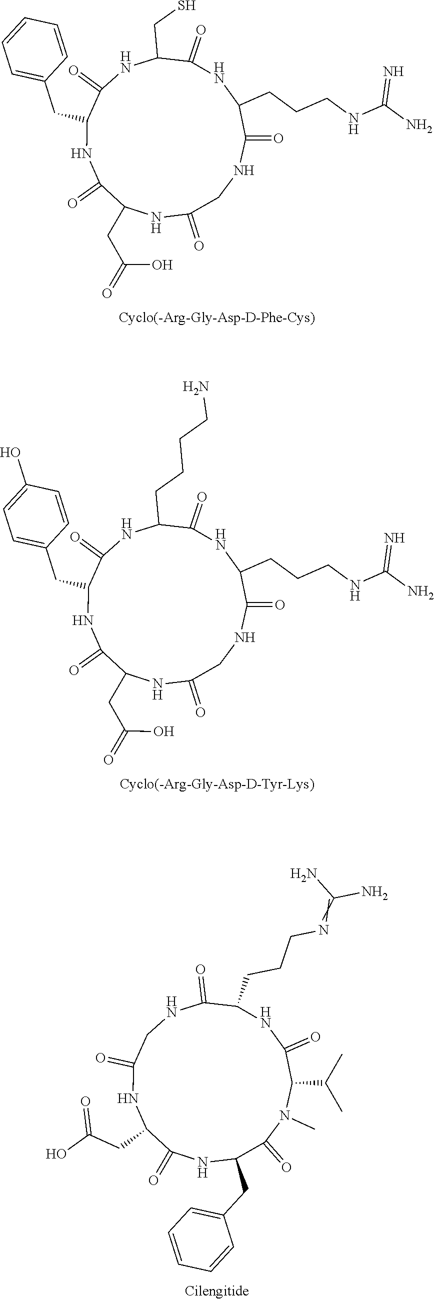 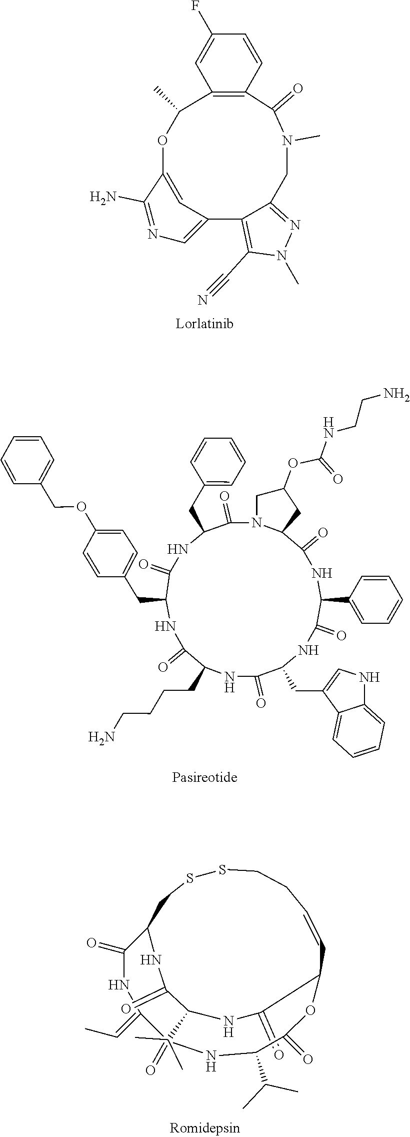
the cyclopeptide having an immunomodulatory activity is one or a combination of the following cyclopeptides: 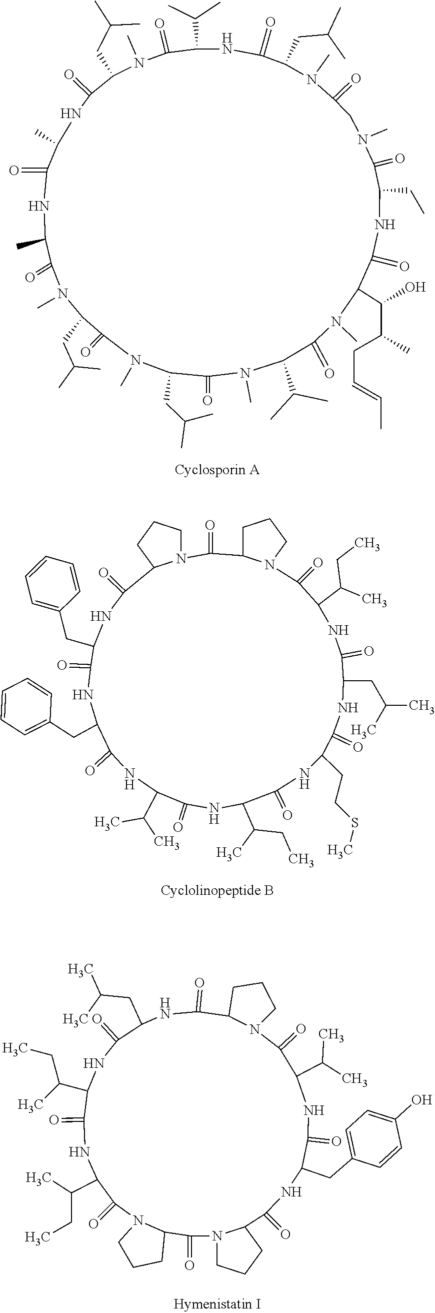
the cyclopeptide having a blood sugar regulating activity is one or a combination of the following cyclopeptides: 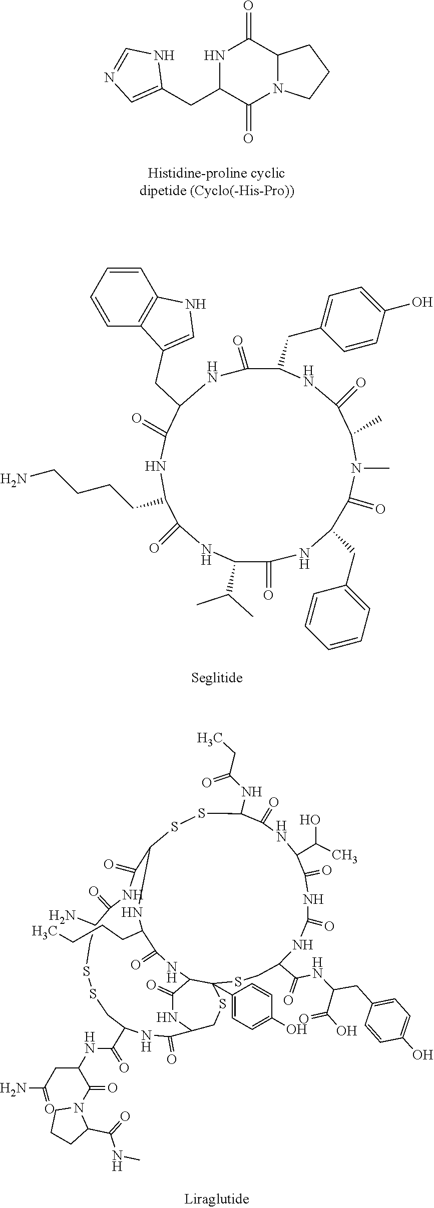
the cyclopeptide having a cardiovascular and blood-related activity is one or a combination of the following cyclopeptides: 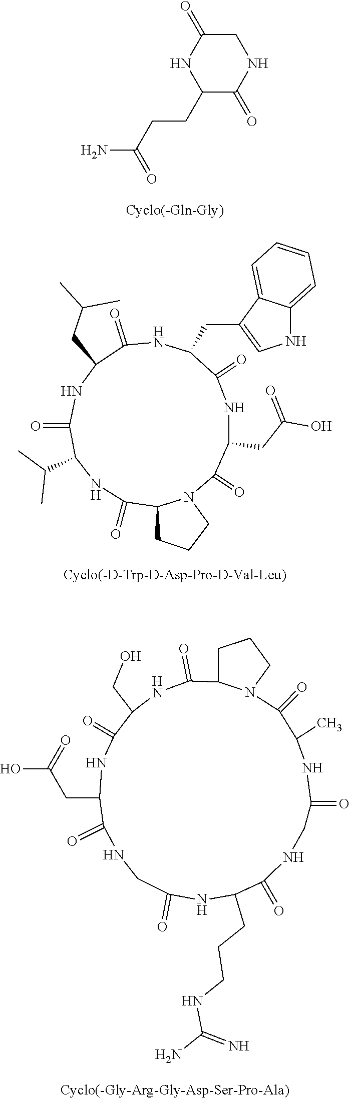 [SMILES: NC(=O)CCC1NC(=O)CNC1=O ; CC(C)C[C@@H]1NC(=O)[C@@H](C(C)C)NC(=O)[C@@H]2CCCN2C(=O)[C@@H](CC(=O)O)NC(=O)[C@@H](Cc2c[nH]c3ccccc23)NC1=O ; CC1NC(=O)CNC(=O)C(CCCNC(=N)N)NC(=O)CNC(=O)C(CC(=O)O)NC(=O)C(CO)NC(=O)C2CCCN2C1=O] 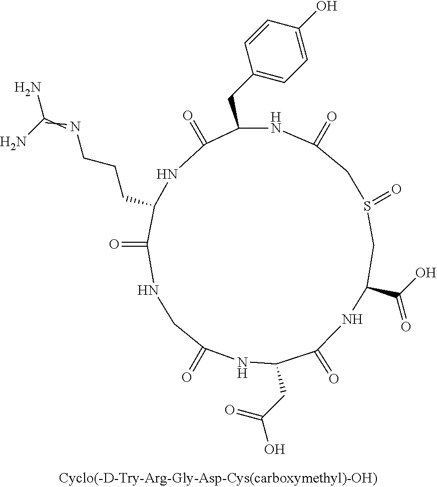 [SMILES: NC(N)=NCCC[C@@H]1NC(=O)[C@@H](Cc2ccc(O)cc2)NC(=O)CS(=O)C[C@@H](C(=O)O)NC(=O)[C@H](CC(=O)O)NC(=O)CNC1=O]
the cyclopeptide having other activities are one or a combination of the following cyclopeptides:

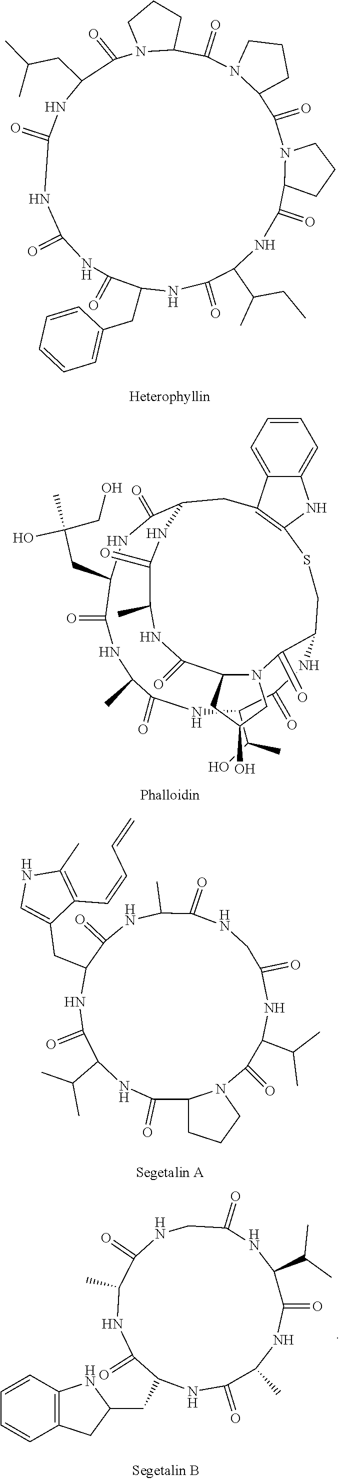

3: The cyclopeptide-based glass according to claim 1, wherein the cyclopeptide comprises a poorly water-soluble cyclopeptide, and a water-soluble cyclopeptide derivative with a modified peptide chain skeleton of a poorly water-soluble cyclopeptide.

4: A pharmaceutical composition comprising the cyclopeptide-based glass according to claim 1, wherein the pharmaceutical composition is in a glass form, and the composition is completely prepared from a cyclopeptide or further comprises pharmaceutical adjuvants and/or active pharmaceutical ingredients.

5: A method for preparing the cyclopeptide-based glass according to claim 1, comprising the following steps:
(1) performing a ball-milling treatment on one or more cyclopeptides to obtain a milled cyclopeptide raw material, wherein a temperature of a raw material during the ball-milling treatment is controlled at 0° C. to 50° C.; and
(2) preparing the cyclopeptide-based glass by a heating-quenching method which comprises heating the milled cyclopeptide raw material to a temperature near a melting point in an inert gas atmosphere, performing heat preservation for a period of time, and transferring to an annealing furnace for annealing treatment.

6: The method according to claim 5, wherein
the temperature of the heating is a temperature of a melting point temperature (Tₘ)±50 to 250 K;
the heat preservation is performed for 0 min to 30 h;
a temperature of the annealing treatment is a temperature of a glass transition temperature (T_{g})±50 to 150 K;
the annealing treatment is performed for 30 min to 2 h; and
Tₘ and T_{g} are measured by thermogravimetric analysis and differential scanning calorimetry, and heating and cooling rates are 2 to 50 K min⁻¹.

7: A method for preparing the pharmaceutical composition according to claim 4, comprising the following steps:
(1) mixing a cyclopeptide with other active pharmaceutical ingredients, and performing a ball-milling treatment on a resulting mixture, wherein a temperature of a raw material during the ball-milling is controlled at 0° C. to 50° C.;
(2) preparing the cyclopeptide-based glass by a “heating-quenching” method which comprises heating a milled cyclopeptide raw material to a temperature near a melting point in an inert gas atmosphere, performing heat preservation for a period of time, and transferring to an annealing furnace for annealing treatment to obtain glass; and
(3) optionally, preparing the glass obtained in step (2) and pharmaceutically acceptable adjuvants into the pharmaceutical composition.

8: A method for preparing the pharmaceutical composition according to claim 4, comprising the following steps:
(1) performing a ball-milling treatment on one or more cyclopeptides to obtain a powder, wherein a temperature of a raw material during the ball-milling is controlled at 0° C. to 50° C.;
(2) heating the powder obtained in step (1) in an inert gas atmosphere, and performing heat preservation for a period of time, wherein a temperature of the heating is a melting point temperature (Tₘ)±50 to 250 K; the heat preservation is performed for 0 min to 30 h;
(3) completely dissolving other active pharmaceutical ingredients in a good solvent and co-solvent;
(4) mixing an active pharmaceutical ingredient solution obtained in step (3) and a molten cyclopeptide in step (1) evenly;
(5) placing a mixture obtained in step (4) at a temperature set in step (2) for rotary evaporation under a reduced pressure to remove a solvent;
(6) transferring a mixture obtained in step (5) to an annealing furnace for annealing treatment, wherein a temperature of the annealing treatment is a glass transition temperature (T_{g})±50 to 150 K, the annealing treatment is performed for 30 min to 2 h; and
(7) optionally, preparing the glass obtained in step (2) and pharmaceutically acceptable adjuvants into the pharmaceutical composition.

9: A method for preparing the pharmaceutical composition according to claim 4, comprising the following steps:
(1) completely dissolving a cyclopeptide and other active pharmaceutical ingredients together in a good solvent and co-solvent;
(2) heating a mixed solution obtained in step (1) in an inert gas atmosphere for rotary evaporation under a reduced pressure to remove a solvent, and then performing heat preservation treatment, wherein a temperature of the heating is a temperature of a melting point temperature (Tₘ)±50 to 250 K, the heat preservation is performed for 0 min to 30 h;
(3) transferring a mixture obtained in step (2) to an annealing furnace for annealing treatment, wherein a temperature of the annealing treatment is a glass transition temperature (T_{g})±50 to 150 K, the annealing treatment is performed for 30 min to 2 h; and
(4) optionally, preparing the glass obtained in step (3) [2)] and pharmaceutically acceptable adjuvants into the pharmaceutical composition.

10: The pharmaceutical composition according to claim 4, wherein the pharmaceutical composition in the glass form is further prepared into an oral preparation, a patch, a subcutaneous embedding agent, a stent material [and] or a microneedle device.

11: The cyclopeptide-based glass according to claim 1, wherein 2≤n≤15.

12: The method according to claim 5, wherein the temperature of the raw material during the ball-milling is controlled at 10° C. to 30° C.

13: The method according to claim 6, wherein the temperature of the heating is a temperature 50 K to 100 K higher than Tₘ.

14: The method according to claim 6, wherein the heat preservation is performed for 15 min to 30 min.

15: The method according to claim 6, wherein the temperature of the annealing treatment is a temperature 50 K to 100 K lower than T_{g}.

16: The method according to claim 6, wherein the annealing treatment is performed for 30 min to 1 h.

17: The method according to claim 7, wherein the temperature of the raw material during the ball-milling is controlled at 10° C. to 30° C.

18: The method according to claim 8, wherein the temperature of the raw material during the ball-milling is controlled at 10° C. to 30° C.

19: The method according to claim 8, wherein the temperature of the heating is a temperature 50 K to 100 K higher than Tₘ, the heat preservation is performed for 15 min to 30 min, the temperature of the annealing treatment is a temperature 50 K to 100 K lower than T_{g}, the annealing treatment is performed for 30 min to 1 h.

20: The method according to claim 9, wherein the temperature of the heating is a temperature 50 K to 100 K higher than Tₘ, the heat preservation is performed for 15 min to 30 min, the temperature of the annealing treatment is a temperature 50 K to 100 K lower than T_{g}, the annealing treatment is performed for 30 min to 1 h.
